SEQUENCE LISTING (117 sequences)

SEQ ID NO: 1 - protein, 5 aa, synthetic construct
  REGION 1..5: note = Description of Artificial Sequence: Synthetic peptide
VPALR

SEQ ID NO: 2 - protein, 5 aa, synthetic construct
  REGION 1..5: note = Description of Artificial Sequence: Synthetic peptide
VSALK

SEQ ID NO: 3 - protein, 6 aa, synthetic construct
  REGION 1..6: note = Description of Artificial Sequence: Synthetic peptide
TVPALR

SEQ ID NO: 4 - protein, 6 aa, synthetic construct
  REGION 1..6: note = Description of Artificial Sequence: Synthetic peptide
TVSALK

SEQ ID NO: 5 - protein, 7 aa, synthetic construct
  REGION 1..7: note = Description of Artificial Sequence: Synthetic peptide
FTVSALK

SEQ ID NO: 6 - protein, 7 aa, synthetic construct
  REGION 1..7: note = Description of Artificial Sequence: Synthetic peptide
LTVSALK

SEQ ID NO: 7 - protein, 7 aa, synthetic construct
  REGION 1..7: note = Description of Artificial Sequence: Synthetic peptide
TFVSALK

SEQ ID NO: 8 - protein, 7 aa, synthetic construct
  REGION 1..7: note = Description of Artificial Sequence: Synthetic peptide
TVSALFK

SEQ ID NO: 9 - protein, 7 aa, synthetic construct
  REGION 1..7: note = Description of Artificial Sequence: Synthetic peptide
TVPALFR

SEQ ID NO: 10 - protein, 7 aa, synthetic construct
  REGION 1..7: note = Description of Artificial Sequence: Synthetic peptide
TVPMLFK

SEQ ID NO: 11 - protein, 7 aa, synthetic construct
  REGION 1..7: note = Description of Artificial Sequence: Synthetic peptide
TVPTLFK

SEQ ID NO: 12 - protein, 6 aa, synthetic construct
  REGION 1..6: note = Description of Artificial Sequence: Synthetic peptide
TVPMLK

SEQ ID NO: 13 - protein, 6 aa, synthetic construct
  REGION 1..6: note = Description of Artificial Sequence: Synthetic peptide
TVPTLK

SEQ ID NO: 14 - protein, 5 aa, synthetic construct
  REGION 1..5: note = Description of Artificial Sequence: Synthetic peptide
VPMLK

SEQ ID NO: 15 - protein, 5 aa, synthetic construct
  REGION 1..5: note = Description of Artificial Sequence: Synthetic peptide
VPTLK

SEQ ID NO: 16 - protein, 6 aa, synthetic construct
  REGION 1..6: note = Description of Artificial Sequence: Synthetic peptide
VPMLKE

SEQ ID NO: 17 - protein, 6 aa, synthetic construct
  REGION 1..6: note = Description of Artificial Sequence: Synthetic peptide
VPTLKD

SEQ ID NO: 18 - protein, 6 aa, synthetic construct
  REGION 1..6: note = Description of Artificial Sequence: Synthetic peptide
VPALRD

SEQ ID NO: 19 - protein, 6 aa, synthetic construct
  REGION 1..6: note = Description of Artificial Sequence: Synthetic peptide
VSALKE

SEQ ID NO: 20 - protein, 6 aa, synthetic construct
  REGION 1..6: note = Description of Artificial Sequence: Synthetic peptide
VSALKD

SEQ ID NO: 21 - protein, 6 aa, synthetic construct
  REGION 1..6: note = Description of Artificial Sequence: Synthetic peptide
TAVSLK

SEQ ID NO: 22 - protein, 6 aa, synthetic construct
  REGION 1..6: note = Description of Artificial Sequence: Synthetic peptide
TALVSK

SEQ ID NO: 23 - protein, 6 aa, synthetic construct
  REGION 1..6: note = Description of Artificial Sequence: Synthetic peptide
TVLSAK

SEQ ID NO: 24 - protein, 6 aa, synthetic construct
  REGION 1..6: note = Description of Artificial Sequence: Synthetic peptide
TLVSAK

SEQ ID NO: 25 - protein, 6 aa, synthetic construct
  REGION 1..6: note = Description of Artificial Sequence: Synthetic peptide
TMVPLK

SEQ ID NO: 26 - protein, 6 aa, synthetic construct
  REGION 1..6: note = Description of Artificial Sequence: Synthetic peptide
TMLVPK

SEQ ID NO: 27 - protein, 6 aa, synthetic construct
  REGION 1..6: note = Description of Artificial Sequence: Synthetic peptide
TVLPMK

SEQ ID NO: 28 - protein, 6 aa, synthetic construct
  REGION 1..6: note = Description of Artificial Sequence: Synthetic peptide
TLVPMK

SEQ ID NO: 29 - protein, 6 aa, synthetic construct
  REGION 1..6: note = Description of Artificial Sequence: Synthetic peptide
TTVPLK

SEQ ID NO: 30 - protein, 6 aa, synthetic construct
  REGION 1..6: note = Description of Artificial Sequence: Synthetic peptide
TTLVPK

SEQ ID NO: 31 - protein, 6 aa, synthetic construct
  REGION 1..6: note = Description of Artificial Sequence: Synthetic peptide
TVLPTK

SEQ ID NO: 32 - protein, 6 aa, synthetic construct
  REGION 1..6: note = Description of Artificial Sequence: Synthetic peptide
TLVPTK

SEQ ID NO: 33 - protein, 6 aa, synthetic construct
  REGION 1..6: note = Description of Artificial Sequence: Synthetic peptide
TAVPLR

SEQ ID NO: 34 - protein, 6 aa, synthetic construct
  REGION 1..6: note = Description of Artificial Sequence: Synthetic peptide
TALVPR

SEQ ID NO: 35 - protein, 6 aa, synthetic construct
  REGION 1..6: note = Description of Artificial Sequence: Synthetic peptide
TVLPAR

SEQ ID NO: 36 - protein, 6 aa, synthetic construct
  REGION 1..6: note = Description of Artificial Sequence: Synthetic peptide
TLVPAR

SEQ ID NO: 37 - protein, 7 aa, synthetic construct
  REGION 1..7: note = Description of Artificial Sequence: Synthetic peptide
TAVSLKE

SEQ ID NO: 38 - protein, 7 aa, synthetic construct
  REGION 1..7: note = Description of Artificial Sequence: Synthetic peptide
TALVSKE

SEQ ID NO: 39 - protein, 7 aa, synthetic construct
  REGION 1..7: note = Description of Artificial Sequence: Synthetic peptide
TVLSAKE

SEQ ID NO: 40 - protein, 7 aa, synthetic construct
  REGION 1..7: note = Description of Artificial Sequence: Synthetic peptide
TLVSAKE

SEQ ID NO: 41 - protein, 7 aa, synthetic construct
  REGION 1..7: note = Description of Artificial Sequence: Synthetic peptide
TMVPLKE

SEQ ID NO: 42 - protein, 7 aa, synthetic construct
  REGION 1..7: note = Description of Artificial Sequence: Synthetic peptide
TMLVPKE

SEQ ID NO: 43 - protein, 7 aa, synthetic construct
  REGION 1..7: note = Description of Artificial Sequence: Synthetic peptide
TVLPMKE

SEQ ID NO: 44 - protein, 7 aa, synthetic construct
  REGION 1..7: note = Description of Artificial Sequence: Synthetic peptide
TLVPMKE

SEQ ID NO: 45 - protein, 7 aa, synthetic construct
  REGION 1..7: note = Description of Artificial Sequence: Synthetic peptide
TTVPLKD

SEQ ID NO: 46 - protein, 7 aa, synthetic construct
  REGION 1..7: note = Description of Artificial Sequence: Synthetic peptide
TTLVPKD

SEQ ID NO: 47 - protein, 7 aa, synthetic construct
  REGION 1..7: note = Description of Artificial Sequence: Synthetic peptide
TVLPTKD

SEQ ID NO: 48 - protein, 7 aa, synthetic construct
  REGION 1..7: note = Description of Artificial Sequence: Synthetic peptide
TLVPTKD

SEQ ID NO: 49 - protein, 7 aa, synthetic construct
  REGION 1..7: note = Description of Artificial Sequence: Synthetic peptide
TAVPLRD

SEQ ID NO: 50 - protein, 7 aa, synthetic construct
  REGION 1..7: note = Description of Artificial Sequence: Synthetic peptide
TALVPRD

SEQ ID NO: 51 - protein, 7 aa, synthetic construct
  REGION 1..7: note = Description of Artificial Sequence: Synthetic peptide
TVLPARD

SEQ ID NO: 52 - protein, 7 aa, synthetic construct
  REGION 1..7: note = Description of Artificial Sequence: Synthetic peptide
TLVPARD

SEQ ID NO: 53 - protein, 7 aa, synthetic construct
  REGION 1..7: note = Description of Artificial Sequence: Synthetic peptide
TAVSLFK

SEQ ID NO: 54 - protein, 7 aa, synthetic construct
  REGION 1..7: note = Description of Artificial Sequence: Synthetic peptide
TALVSFK

SEQ ID NO: 55 - protein, 7 aa, synthetic construct
  REGION 1..7: note = Description of Artificial Sequence: Synthetic peptide
TVLSAFK

SEQ ID NO: 56 - protein, 7 aa, synthetic construct
  REGION 1..7: note = Description of Artificial Sequence: Synthetic peptide
TLVSAFK

SEQ ID NO: 57 - protein, 7 aa, synthetic construct
  REGION 1..7: note = Description of Artificial Sequence: Synthetic peptide
TMVPLFK

SEQ ID NO: 58 - protein, 7 aa, synthetic construct
  REGION 1..7: note = Description of Artificial Sequence: Synthetic peptide
TMLVPFK

SEQ ID NO: 59 - protein, 7 aa, synthetic construct
  REGION 1..7: note = Description of Artificial Sequence: Synthetic peptide
TVLPMFK

SEQ ID NO: 60 - protein, 7 aa, synthetic construct
  REGION 1..7: note = Description of Artificial Sequence: Synthetic peptide
TLVPMFK

SEQ ID NO: 61 - protein, 7 aa, synthetic construct
  REGION 1..7: note = Description of Artificial Sequence: Synthetic peptide
TTVPLFK

SEQ ID NO: 62 - protein, 7 aa, synthetic construct
  REGION 1..7: note = Description of Artificial Sequence: Synthetic peptide
TTLVPFK

SEQ ID NO: 63 - protein, 7 aa, synthetic construct
  REGION 1..7: note = Description of Artificial Sequence: Synthetic peptide
TVLPTFK

SEQ ID NO: 64 - protein, 7 aa, synthetic construct
  REGION 1..7: note = Description of Artificial Sequence: Synthetic peptide
TLVPTFK

SEQ ID NO: 65 - protein, 7 aa, synthetic construct
  REGION 1..7: note = Description of Artificial Sequence: Synthetic peptide
TAVPLFR

SEQ ID NO: 66 - protein, 7 aa, synthetic construct
  REGION 1..7: note = Description of Artificial Sequence: Synthetic peptide
TALVPFR

SEQ ID NO: 67 - protein, 7 aa, synthetic construct
  REGION 1..7: note = Description of Artificial Sequence: Synthetic peptide
TVLPAFR

SEQ ID NO: 68 - protein, 7 aa, synthetic construct
  REGION 1..7: note = Description of Artificial Sequence: Synthetic peptide
TLVPAFR

SEQ ID NO: 69 - protein, 8 aa, Homo sapiens
KFTVPMLK

SEQ ID NO: 70 - protein, 8 aa, Mus sp.
KLTVPTLK

SEQ ID NO: 71 - protein, 8 aa, Rattus sp.
KFTVPALR

SEQ ID NO: 72 - protein, 8 aa, synthetic construct
  REGION 1..8: note = Description of Artificial Sequence: Synthetic peptide
KFTVSALK

SEQ ID NO: 73-80 - skipped (8 entries)

SEQ ID NO: 81 - protein, 6 aa, synthetic construct
  REGION 1..6: note = Description of Artificial Sequence: Synthetic peptide
  MOD_RES 3: note = S or P
  MOD_RES 4: note = A, M, or T
  REGION 1..6: note = See specification as filed for detailed description of substitutions and preferred embodiments
TVXXLK

SEQ ID NO: 82 - protein, 7 aa, synthetic construct
  REGION 1..7: note = Description of Artificial Sequence: Synthetic peptide
  MOD_RES 3: note = S or P
  MOD_RES 4: note = A, M, or T
  REGION 1..7: note = See specification as filed for detailed description of substitutions and preferred embodiments
TVXXLFK

SEQ ID NO: 83 - protein, 6 aa, synthetic construct
  REGION 1..6: note = Description of Artificial Sequence: Synthetic peptide
KLASVT

SEQ ID NO: 84 - protein, 7 aa, synthetic construct
  REGION 1..7: note = Description of Artificial Sequence: Synthetic peptide
KFLASVT

SEQ ID NO: 85 - protein, 736 aa, Adeno-associated virus
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 86 - protein, 609 aa, Homo sapiens
MSGWESYYKTEGDEEAEEEQEENLEASGDYKYSGRDSLIFLVDASKAMFESQSEDELTPFDMSIQCIQSVYISKIISSDRDLLAVVFYGTEKDKNSVNFKNIYVLQELDNPGAKRILELDQFKGQQGQKRFQDMMGHGSDYSLSEVLWVCANLFSDVQFKMSHKRIMLFTNEDNPHGNDSAKASRARTKAGDLRDTGIFLDLMHLKKPGGFDISLFYRDIISIAEDEDLRVHFEESSKLEDLLRKVRAKETRKRALSRLKLKLNKDIVISVGIYNLVQKALKPPPIKLYRETNEPVKTKTRTFNTSTGGLLLPSDTKRSQIYGSRQIILEKEETEELKRFDDPGLMLMGFKPLVLLKKHHYLRPSLFVYPEESLVIGSSTLFSALLIKCLEKEVAALCRYTPRRNIPPYFVALVPQEEELDDQKIQVTPPGFQLVFLPFADDKRKMPFTEKIMATPEQVGKMKAIVEKLRFTYRSDSFENPVLQQHFRNLEALALDLMEPEQAVDLTLPKVEAMNKRLGSLVDEFKELVYPPDYNPEGKVTKRKHDNEGSGSKRPKVEYSEEELKTHISKGTLGKFTVPMLKEACRAYGLKSGLKKQELLEALTKHFQD

SEQ ID NO: 87 - protein, 608 aa, Mus sp.
MSEWESYYKTEGEEEEEEEESPDTGGEYKYSGRDSLIFLVDASRAMFESQGEDELTPFDMSIQCIQSVYTSKIISSDRDLLAVVFYGTEKDKNSVNFKNIYVLQDLDNPGAKRVLELDQFKGQQGKKHFRDTVGHGSDYSLSEVLWVCANLFSDVQLKMSHKRIMLFTNEDDPHGRDSAKASRARTKASDLRDTGIFLDLMHLKKPGGFDVSVFYRDIITTAEDEDLGVHFEESSKLEDLLRKVRAKETKKRVLSRLKFKLGEDVVLMVGIYNLVQKANKPFPVRLYRETNEPVKTKTRTFNVNTGSLLLPSDTKRSLTYGTRQIVLEKEETEELKRFDEPGLILMGFKPTVMLKKQHYLRPSLFVYPEESLVSGSSTLFSALLTKCVEKEVIAVCRYTPRKNVSPYFVALVPQEEELDDQNIQVTPGGFQLVFLPYADDKRKVPFTEKVTANQEQIDKMKAIVQKLRFTYRSDSFENPVLQQHFRNLEALALDMMESEQVVDLTLPKVEAIKKRLGSLADEFKELVYPPGYNPEGKVAKRKQDDEGSTSKKPKVELSEEELKAHFRKGTLGKLTVPTLKDICKAHGLKSGPKKQELLDALIRHLEKN

SEQ ID NO: 88 - protein, 608 aa, Rattus sp.
MSEWESYYKTEGEEEEEEEQSPDTNGEYKYSGRDSLIFLVDASRAMFESQGEDELTPFDMSIQCIQSVYTSKIISSDRDLLAVVFYGTEKDKNSVNFKSIYVLQDLDNPGAKRVLELDRFKGQQGKKHFRDTIGHGSDYSLSEVLWVCANLFSDVQFKMSHKRIMLFTNEDDPHGNDSAKASRARTKASDLRDTGIFLDLMHLKKRGGFDVSLFYRDIISIAEDEDLGVHFEESSKLEDLLRKVRAKETKKRVLSRLKFKLGKDVALMVGVYNLVQKANKPFPVRLYRETNEPVKTKTRTFNVNTGSLLLPSDTKRSLTFGTRQIVLEKEETEELKRFDEPGLILMGFKPMVMLKNHHYLRPSLFLYPEESLVNGSSTLFSALLTKCVEKEVIAVCRYTARKNVSPYFVALVPQEEELDDQNIQVTPAGFQLVFLPYADDKRKVPFTEKVMANPEQIDKMKAIVQKLRFTYRSDSFENPVLQQHFRNLEALALDMMESEQVVDLTLPKVEAIKKRLGSLADEFKELVYPPGYNPEGKIAKRKADNEGSASKKPKVELSEEELKDLFAKGTLGKLTVPALRDICKAYGLKSGPKKQELLEALSRHLEKN

SEQ ID NO: 89 - protein, 742 aa, synthetic construct
  REGION 1..742: note = Description of Artificial Sequence: Synthetic polypeptide
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQTVSALKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 90 - protein, 743 aa, synthetic construct
  REGION 1..743: note = Description of Artificial Sequence: Synthetic polypeptide
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQTVSALFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 91 - DNA, 21 bp, synthetic construct
  misc_feature 1..21: note = Description of Artificial Sequence: Synthetic primer
  source 1..21: mol_type = other DNA
ggaacccctagtgatggagtt

SEQ ID NO: 92 - DNA, 16 bp, synthetic construct
  misc_feature 1..16: note = Description of Artificial Sequence: Synthetic primer
  source 1..16: mol_type = other DNA
cggcctcagtgagcga

SEQ ID NO: 93 - protein, 18 aa, synthetic construct
  REGION 1..18: note = Description of Artificial Sequence: Synthetic peptide
RGGRLSYSRRRFSTSTGR

SEQ ID NO: 94 - protein, 20 aa, synthetic construct
  REGION 1..20: note = Description of Artificial Sequence: Synthetic peptide
HARIKPTFRRLKWKYKGKFW

SEQ ID NO: 95 - protein, 12 aa, synthetic construct
  REGION 1..12: note = Description of Artificial Sequence: Synthetic peptide
PLSSIFSRIGDP

SEQ ID NO: 96 - protein, 21 aa, synthetic construct
  REGION 1..21: note = Description of Artificial Sequence: Synthetic peptide
AGYLLGKINLKALAALAKKIL

SEQ ID NO: 97 - protein, 18 aa, synthetic construct
  REGION 1..18: note = Description of Artificial Sequence: Synthetic peptide
VRLPPPVRLPPPVRLPPP

SEQ ID NO: 98 - protein, 18 aa, synthetic construct
  REGION 1..18: note = Description of Artificial Sequence: Synthetic peptide
VELPPPVELPPPVELPPP

SEQ ID NO: 99 - protein, 16 aa, synthetic construct
  REGION 1..16: note = Description of Artificial Sequence: Synthetic peptide
KGTYKKKLMRIPLKGT

SEQ ID NO: 100 - protein, 9 aa, synthetic construct
  REGION 1..9: note = Description of Artificial Sequence: Synthetic peptide
PPRPPRPPR

SEQ ID NO: 101 - protein, 15 aa, synthetic construct
  REGION 1..15: note = Description of Artificial Sequence: Synthetic peptide
PPRPPRPPRPPRPPR

SEQ ID NO: 102 - protein, 8 aa, synthetic construct
  REGION 1..8: note = Description of Artificial Sequence: Synthetic peptide
RRRRRRRR

SEQ ID NO: 103 - protein, 16 aa, synthetic construct
  REGION 1..16: note = Description of Artificial Sequence: Synthetic peptide
LRRERQSRLRRERQSR

SEQ ID NO: 104 - protein, 9 aa, synthetic construct
  REGION 1..9: note = Description of Artificial Sequence: Synthetic peptide
RKKRRQRRR

SEQ ID NO: 105 - protein, 524 aa, synthetic construct
  REGION 1..524: note = Description of Artificial Sequence: Synthetic polypeptide
METDTLLLWVLLLWVPGSTGDYPYDVPDYAGAQPADDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRGGGGSGGGGSGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSAVDEAKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDEQKLISEEDLN

SEQ ID NO: 106 - DNA, 1575 bp, synthetic construct
  misc_feature 1..1575: note = Description of Artificial Sequence: Synthetic polynucleotide
  source 1..1575: mol_type = other DNA
atggagacagacacactcctgctatgggtactgctgctctgggttccaggttccactggtgactatccatatgatgttccagattatgctggggcccagccggccgacgacatccaaatgacccagagtccatctagtctgtctgcttcggtaggtgatagggtcactattacttgcagggcctcccaggacgtgtcaactgcagtggcttggtaccaacagaagcccgggaaagctcccaaactgctgatctactccgccagctttctgtattccggagttccgtctagattttccggatcaggaagcggcacggatttcacactcacaataagcagcctacaaccagaggacttcgcaacctactattgtcaacagtacctgtaccatccagccacctttgggcagggcaccaaggtggaaatcaagcgcggtggtggtggatcaggtggaggcggaagtggaggtggcggatccgaagttcagcttgtcgagtccggtggcggcctggttcagcctggtgggtctttgcgtctgtcatgcgccgcctctggtttcaccttttcagactcttggatccactgggtgagacaggccccaggaaagggtcttgagtgggttgcatggattagcccctacggcggcagtacatattacgcggatagcgtgaaagggaggtttaccatcagcgcagacacgtcgaagaacaccgcatacctccagatgaattccctccgagccgaagataccgccgtgtactattgtgctcgccggcattggcctggcggcttcgattattggggacagggaactctagtaacagtgtcggctgtcgacgaggccaaatcttgtgacaaaactcacacatgcccaccgtgcccagcacccgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaggggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaagtcgacgaacaaaaactcatctcagaagaagatctgaattga

SEQ ID NO: 107 - protein, 25 aa, Homo sapiens
MGVLLTQRTLLSLVLALLFPSMASM

SEQ ID NO: 108 - protein, 19 aa, Homo sapiens
MGWSCIILFLVATATGVHS

SEQ ID NO: 109 - protein, 21 aa, Homo sapiens
MWWRLWWLLLLLLLLWPMVWA

SEQ ID NO: 110 - protein, 22 aa, Homo sapiens
MDMRVPAQLLGLLLLWLRGARC

SEQ ID NO: 111 - protein, 16 aa, Homo sapiens
MPLLLLLPLLWAGALA

SEQ ID NO: 112 - protein, 23 aa, Homo sapiens
MDAMKRGLCCVLLLCGAVFVSPS

SEQ ID NO: 113 - protein, 18 aa, Homo sapiens
MAFLWLLSCWALLGTTFG

SEQ ID NO: 114 - protein, 15 aa, Homo sapiens
MNLLLILTFVAAAVA

SEQ ID NO: 115 - protein, 20 aa, Homo sapiens
MYRMQLLSCIALSLALVTNS

SEQ ID NO: 116 - protein, 16 aa, Homo sapiens
MKWVTFISLLFSSAYS

SEQ ID NO: 117 - protein, 24 aa, Homo sapiens
MALWMRLLPLLALLALWGPDPAAA

(19) United States
(12) Patent Application Publication
(10) Pub. No.: US 2025/0074949 A1
(43) Pub. Date: Mar. 6, 2025

(54) METHODS AND COMPOSITIONS FOR DELIVERY OF IMMUNOTHERAPY AGENTS ACROSS THE BLOOD-BRAIN BARRIER TO TREAT BRAIN CANCER

(71) Applicant: The Brigham and Women's Hospital, Inc., Boston, MA (US)

(72) Inventors: Fengfeng Bei, West Roxbury, MA (US); E. Antonio Chiocca, Wakefield, MA (US)

(21) Appl. No.: 18/633,827

(22) Filed: Apr. 12, 2024

Related U.S. Application Data
(62) Division of application No. 17/791,261, filed on Jul. 7, 2022, now Pat. No. 11,981,705.
(60) Provisional application No. 62/959,625, filed on Jan. 10, 2020.

(51) Int. Cl. C07K 14/005; A61K 31/175; A61K 31/495; A61K 39/00; A61K 39/395; A61P 25/00; A61P 35/00; A61P 37/04; C07K 16/28

(52) U.S. Cl. CPC C07K 14/005; A61K 31/175; A61K 31/495; A61K 39/00; A61K 39/0011; A61K 39/3955; A61P 25/00; A61P 35/00; A61P 37/04; C07K 16/2818; C07K 16/2827; A61K 2039/505; A61K 2039/54; C12N 2750/14122; C12N 2750/14143; C12N 2750/14171

(57) ABSTRACT

The present application relates to sequences that enhance permeation of immunotherapy agents across the blood brain barrier, compositions comprising the sequences, and methods of use thereof to treat brain cancer, e.g., glioblastoma (GBM).

### CLAIM OF PRIORITY

This application is a divisional of U.S. patent application Ser. No. 17/791,261, filed Jul. 7, 2022, which is a U.S. National Phase Application under 35 U.S.C. § 371 of International Patent Application No. PCT/US2021/012746, filed on Jan. 8, 2021, which claims the benefit of U.S. Provisional Application Ser. No. 62/959,625, filed on Jan. 10, 2020. The entire contents of the foregoing are incorporated herein by reference.

### SEQUENCE LISTING

This application contains a Sequence Listing that has been submitted electronically as an XML file named “40978-0035002_SL_ST26.XML.” The XML file, created on Apr. 11, 2024, is 142,752 bytes in size. The material in the XML file is hereby incorporated by reference in its entirety.

### TECHNICAL FIELD

Described herein are sequences that enhance permeation of immunotherapy agents across the blood brain barrier, compositions comprising the sequences, and methods of use thereof to treat brain cancer, e.g., glioblastoma (GBM).

### BACKGROUND

Glioblastoma multiforme (GBM) is the most common and deadly brain tumor in adults with a median overall survival of only 15 months¹. Approximately 12,000 new GBM cases are diagnosed every year in the United States with an incidence rate of 3.2 per 100,000 population². Despite significant progress made in understanding the histology, molecular landscape and tumor microenvironment of GBM³⁻⁶, there have been few therapeutic advances since 2005. One critical obstacle in turning our wealth of knowledge on GBM into effective therapy is the inefficient drug delivery to the GBM tumor site. Intravenous administration is a convenient and widely applicable route of drug administration that, in theory, could achieve good tumor coverage as GBM tumor is well vascularized structurally⁷. However, designing drugs that cross the blood-brain barrier (BBB) and/or blood-tumor barrier remains challenging.

### SUMMARY

Glioblastoma is an extremely deadly brain cancer that is difficult to treat using conventional methods. Systemically administered cancer gene therapy is a new treatment paradigm for tackling glioblastoma. Described herein are brain-penetrant AAV viral vectors engineered to establish an intravascular gene delivery platform for glioblastoma gene therapy, e.g., to systemically delivers PD-L1 antibodies for the treatment of glioblastoma.

Thus, provided herein are methods for delivering an immunotherapy agent to a cancer in a subject. The methods include administering to the subject an adeno-associated virus (AAV) comprising (i) a capsid protein comprising an amino acid sequence that comprises at least four contiguous amino acids from the sequence TVSALFK (SEQ ID NO:8); TVSALK (SEQ ID NO:4); KLASVT (SEQ ID NO:83); or KFLASVT (SEQ ID NO:84), and (ii) a transgene encoding an immunotherapy agent, optionally wherein the cancer cell is in the brain of a human subject.

In some embodiments, the amino acid sequence comprises at least five contiguous amino acids from the sequence TVSALK (SEQ ID NO:4); TVSALFK (SEQ ID NO:8); KLASVT (SEQ ID NO:83); or KFLASVT (SEQ ID NO:84).

In some embodiments, the amino acid sequence comprises at least six contiguous amino acids from the sequence TVSALK (SEQ ID NO:4); TVSALFK (SEQ ID NO:8); KLASVT (SEQ ID NO:83); or KFLASVT (SEQ ID NO:84).

Also provided herein are methods for delivering an immunotherapy agent to a cancer in a subject. The methods include administering to the subject an adeno-associated virus (AAV) comprising (i) a capsid protein comprising an amino acid sequence that comprises at least four contiguous amino acids from the sequence V[S/p][A/m/t/]L (SEQ ID NO:79), TV[S/p][A/m/t/]L (SEQ ID NO:80), TV[S/p][A/m/t/]LK (SEQ ID NO:81), or TV[S/p][A/m/t/]LFK. (SEQ ID NO:82), and (ii) a transgene encoding an immunotherapy agent, optionally wherein the cancer cell is in the brain of a human subject.

In some embodiments, the targeting sequence comprises VPALR (SEQ ID NO:1); VSALK (SEQ ID NO:2); TVPALR (SEQ ID NO:3); TVSALK (SEQ ID NO:4); TVPMLK (SEQ ID NO: 12); TVPTLK (SEQ ID NO:13); FTVSALK (SEQ ID NO:5); LTVSALK (SEQ ID NO:6); TVSALFK (SEQ ID NO:8); TVPALFR (SEQ ID NO:9); TVPMLFK (SEQ ID NO:10) or TVPTLFK (SEQ ID NO:11).

In some embodiments, the transgene encoding an immunotherapy agent encodes an antibody targeting PD-1 or PD-L1.

In some embodiments, the subject is a mammalian subject.

In some embodiments, the AAV is AAV9.

In some embodiments, the AAV9 comprises AAV9 VP1.

In some embodiments, the targeting sequence is inserted in a position corresponding to amino acids 588 and 589 of AAV9 VP1 comprising SEQ ID NO:85.

In some embodiments, the cell is in the brain of the subject, and the AAV is administered by parenteral delivery; intracerebral; or intrathecal delivery.

In some embodiments, the parenteral delivery is via intravenous, intraarterial, subcutaneous, intraperitoneal, or intramuscular delivery.

In some embodiments, the intrathecal delivery is via lumbar injection, cisternal magna injectio, or intraparenchymal injection.

In some embodiments, the methods further include administering chemotherapy, radiation, and/or surgical resection to the subject.

In some embodiments, the chemotherapy comprises temozolamide, lomustine, or a combination thereof.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-1C depict an exemplary strategy of engineering AAV9 by inserting cell-penetrating peptides (CPPs) into its capsid. FIG. 1A is a 3D model of an AAV9 virus. Individual CPP inserted into the capsid between amino acids 588 and 589 (VP1 numbering) will be displayed at the 3-fold axis where receptor binding presumably occurs. FIG. 1B illustrates the method of individual AAV production. Three plasmids including pRC (engineered or not), pHelper and pAAV are co-transfected into HEK 293T cells, with AAVs harvested and purified using iodixanol gradient. FIG. 1C is a vector diagram of an exemplary vector comprising a sequence encoding an anti-PDL1 antibody.

FIGS. 2A-2B depict representative images of mouse brain sections (FIG. 2A) and their quantitative analysis (FIG. 2B) after intravenous administration of low-dose candidate AAVs. Mice with mixed genetic background are used. Candidate AAVs differs in their inserted CPPs (see Table 3), but all express nuclear red fluorescent protein (RFP) as reporter. Candidate AAVs with low production yields are excluded for further screening. The dose of AAV is 1×10¹⁰ vg (viral genome) per animal. Each white dot in FIG. 2A represents a RFP-labeled cell. In FIG. 2B, * P<0.05, vs. AAV9, ANOVA.

FIGS. 2C-2D depict representative images of mouse brain sections (FIG. 2C) and their quantitative analysis (FIG. 2D) after intravenous administration of AAV.CPP.11 and AAV.CPP.12 in a repeat experiment. AAV.CPP.11 and AAV.CPP.12 contain CPPs BIP1 and BIP2 respectively (see Table 3). The doses of the AAVs are increased to 1×10¹¹ vg per animal. Candidate AAVs express nuclear red fluorescent protein (RFP) as reporter. Each white dot in FIG. 2C represents a RFP-labeled cell. In FIG. 2D, * P<0.05, ** P<0.01, vs. AAV9, ANOVA.

FIG. 3A depicts the optimization of the BIP targeting sequence in order to further engineer AAV9 towards better brain transduction. BIP1 (VPALR, SEQ ID NO:1), which enables AAV9 to transduce brain more efficiently (as in AAV.CPP.11), is derived from the protein Ku70 in rats. Human, mouse and rat Ku70 proteins differ in their exact amino acid sequences. BIP2 (VSALK, SEQ ID NO:2) as in AAV.CPP.12 is a “synthetic” peptide related to BIP1. Further engineering focuses on the VSALK sequence in the hope of minimizing species specificity of final engineered AAV To generate new targeting sequence, amino acids of interest are added to the VSALK sequence, and in other cases positions of individual amino acids are switched. All new BIP2-derived sequences are again inserted into the AAV9 capsid to generate new candidate AAVs for screening. Sequences appearing in order are SEQ ID NOs: 69, 70, 71, 1-6, 72, 7, and 8.

FIGS. 3B-3C depict representative images of mouse brain sections (FIG. 3B) and their quantitative analysis (FIG. 3C) after intravenous administration of more candidate AAVs. All candidate AAVs express nuclear red fluorescent protein (RFP) as reporter. The dose of AAV is 1×10¹¹ vg per animal. Each white dot in FIG. 3B represents a RFP-labeled cell. AAV.CPP.16 and AAV.CPP.21 were identified as top hits with their robust and widespread brain transduction. In FIG. 3C, * P<0.05, ** P<0.01, *** P<0.001, vs. AAV9, ANOVA.

FIG. 3D depicts quantitative analysis of transduction efficiency in the liver after intravenous administration of candidate AAVs. Percentage of transduced liver cells is presented. The dose of AAV is 1×10¹¹ vg per animal. *** P<0.001, vs. AAV9, ANOVA.

FIGS. 4A-4E depict screening of selected candidate AAVs in an in vitro spheroid model of human blood-brain barrier. FIG. 4A illustrates the spheroid comprising human microvascular endothelial cells, which forms a barrier at the surface, and human pericyte and astrocytes inside the spheroid. Candidate AAVs were assessed for their ability to penetrate from the surrounding medium into the inside of the spheroid and to transduce the cells inside. FIG. 4B-4D shows images of AAV9 (FIG. 4B), AAV.CPP.16 (FIG. 4C) and AAV.CPP.21 (FIG. 4D) treated spheroids. FIG. 4E shows relative RFP intensity of different AAV treated spheroids. *** P<0.001, vs. AAV9, ANOVA.

FIGS. 5A-5B depict representative images of brain sections (FIG. 5A) and their quantitative analysis (FIG. 5B) after intravenous administration of AAV9, AAV.CPP.16 and AAV.CPP.21 in C57BL/6J inbred mice. All candidate AAVs express nuclear red fluorescent protein (RFP) as reporter. The dose of AAV is 1×10¹² vg per animal. Each white dot in FIG. 5A represents a RFP-labeled cell. In FIG. 5B, * P<0.05, *** P<0.001, ANOVA.

FIGS. 6A-6B depict representative images of brain sections (FIG. 6A) and their quantitative analysis (FIG. 6B) after intravenous administration of AAV9, AAV.CPP.16 and AAV.CPP.21 in BALB/cJ inbred mice. All candidate AAVs express nuclear red fluorescent protein (RFP) as reporter. The dose of AAV is 1×10¹² vg per animal. Each white dot in FIG. 6A represents a RFP-labeled cell. In FIG. 6B, *** P<0.001, ANOVA.

FIGS. 7A-7B depict representative images of brain sections (FIG. 7A) and their quantitative analysis (FIG. 7B) after intravenous administration of high-dose AAV.CPP.16 and AAV.CPP.21 in C57BL/6J inbred mice. Both candidate AAVs express nuclear red fluorescent protein (RFP) as reporter. The dose of AAV is 4×10¹² vg per animal. Each white dot in FIG. 7A represents a RFP-labeled cell. In FIG. 7B, * P<0.05, Student test.

FIG. 8A shows AAV.CPP.16 and AAV.CPP.21 transduce adult neurons (labeled by a NeuN antibody) across multiple brain regions in mice including the cortex, midbrain and hippocampus. Transduced neurons are co-labeled by NeuN antibody and RFP. AAVs of 4×10¹² vg were administered intravenously in adult C57BL/6J mice (6 weeks old).

FIG. 8B depicts that AAV.CPP.16 and AAV.CPP.21 show enhanced ability vs. AAV9 in targeting the spinal cord and motor neurons in mice. AAVs of 4×10¹⁰ vg were administered intravenously into neonate mice (1 day after birth). Motor neurons in the ventral horn of the spinal cord were visualized using CHAT antibody staining. Co-localization of RFP and CHAT signals suggests specific transduction of the motor neurons.

FIG. 9A depicts that AAV.CPP.16 shows enhanced ability vs. AAV9 in targeting the heart in adult mice. AAVs of 1×10¹¹ vg were administered intravenously in adult C57BL/6J mice (6 weeks old). Percentage of RFP-labeled cells relative to all DAPI-stained cells is presented. * P<0.05, Student test.

FIG. 9B depicts that AAV.CPP.16 shows enhanced ability vs. AAV9 in targeting the skeletal muscle in adult mice. AAVs of 1×10¹¹ vg were administered intravenously in adult C57BL/6J mice (6 weeks old). Percentage of RFP-labeled cells relative to all DAPI-stained cells is presented. * P<0.05, Student test.

FIG. 9C depicts that AAV.CPP.16 shows enhanced ability vs. AAV9 in targeting the dorsal root ganglion (DRG) in adult mice. AAVs of 1×10¹¹ vg were administered intravenously in adult C57BL/6J mice (6 weeks old). Percentage of RFP-labeled cells relative to all DAPI-stained cells is presented. * P<0.05, Student test.

FIG. 10A depicts that AAV.CPP.16 and AAV.CPP.21 show enhanced ability vs. AAV9 to transduce brain cells in primary visual cortex after intravenous administration in non-human primates. 2×10¹³ vg/kg AAVs-CAG-AADC (as reporter gene) were injected intravenously into 3 months old cynomolgus monkeys with low pre-existing neutralizing antibody. AAV-transduced cells (shown in black) were visualized using antibody staining against AADC. Squared areas in the left panels are enlarged as in the right panels. AAV.CPP.16 transduced significantly more cells vs. AAV9. AAV.CPP.21 also transduced more cell vs. AAV9 although its effect was less evident in comparison with AAV.CPP.16.

FIG. 10B depicts that AAV.CPP.16 and AAV.CPP.21 show enhanced ability vs. AAV9 to transduce brain cells in parietal cortex after intravenous administration in non-human primates. 2×10¹³ vg/kg AAVs-CAG-AADC (as reporter gene) were injected intravenously into 3 months old cynomolgus monkeys with low pre-existing neutralizing antibody. AAV-transduced cells (shown in black) were visualized using antibody staining against AADC. Squared areas in the left panels are enlarged as in the right panels. AAV.CPP.16 transduced significantly more cells vs. AAV9. AAV.CPP.21 also transduced more cell vs. AAV9 although its effect was less evident in comparison with AAV.CPP.16.

FIG. 10C depicts that AAV.CPP.16 and AAV.CPP.21 show enhanced ability vs. AAV9 to transduce brain cells in thalamus after intravenous administration in non-human primates. 2×10¹³ vg/kg AAVs-CAG-AADC (as reporter gene) were injected intravenously into 3 months old cynomolgus monkeys with low pre-existing neutralizing antibody. AAV-transduced cells (shown in black) were visualized using antibody staining against AADC. Squared areas in the left panels are enlarged as in the right panels. AAV.CPP.16 transduced significantly more cells vs. AAV9. AAV.CPP.21 also transduced more cell vs. AAV9 although its effect was less evident in comparison with AAV.CPP.16.

FIG. 10D depicts that AAV.CPP.16 and AAV.CPP.21 show enhanced ability vs. AAV9 to transduce brain cells in cerebellum after intravenous administration in non-human primates. 2×10¹³ vg/kg AAVs-CAG-AADC (as reporter gene) were injected intravenously into 3 months old cynomolgus monkeys with low pre-existing neutralizing antibody. AAV-transduced cells (shown in black) were visualized using antibody staining against AADC. Squared areas in the left panels are enlarged as in the right panels. Both AAV.CPP.16 and AAV.CPP.21 transduced significantly more cells vs. AAV9.

FIGS. 11A-11B depict that AAV.CPP.16 and AAV.CPP.21 do not bind to LY6A. LY6A serves as a receptor for AAV.PHP.B and its variants including AAV.PHP.eB (as in U.S. Pat. No. 9,102,949, US20170166926) and mediates AAV.PHP.eB's robust effect in crossing the BBB in certain mouse strains (Hordeaux et al. Mol Ther 2019 27(5):912-921; Huang et al. 2019, dx.doi.org/10.1101/538421). Over-expressing mouse LY6A in cultured 293 cells significantly increases binding of AAV.PHP.eB to the cell surface (FIG. 11A). On the contrary, over-expressing LY6A does not increase viral binding for AAV9, AAV.CPP.16 or AAV.CPP.21 (FIG. 11B). This suggests AAV.CPP.16 or AAV.CPP.21 does not share LY6A with AAV.PHP.eB as a receptor.

FIGS. 12A-12C depict that AAV.CPP.21 can be used to systemically deliver a therapeutic gene into brain tumor in a mouse mode of glioblastoma (GBM). As in FIG. 11A, intravenously administered AAV.CPP.21-H2BmCherry was shown to target tumor mass, especially the tumor expanding frontier (FIG. 12A). In FIG. 11B (images) and FIG. 11C (quantitative analysis), using AAV.CPP.21 to systemically deliver the “suicide gene” HSV.TK1 results in shrinkage of brain tumor mass, when combined with the pro-drug ganciclovir. HSV.TK1 turns the otherwise “dormant” ganciclovir into a tumor-killing drug. * P<0.05, Student test.

FIG. 13 depicts that when injected locally into adult mouse brain, AAV.CPP.21 resulted in more widespread and robust transduction of brain tissue in comparison with AAV9. Intracerebral injection of AAVs (1×10¹¹ vg) was performed in adult mice (>6 weeks old) and brain tissues were harvested and examined 3 weeks after AAV injection. ** P<0.01, Student test.

FIG. 14 is a set of images comparing delivery efficiency to the GBM tumor microenvironment in a mouse model using AAV9 (top) and AAV.CPP16 (bottom). As can be seen in the insets (right), AAV.CPP16 provided greater delivery efficacy.

FIGS. 15A-C show that AAV.CPP.16-antiPD-L1 mediated immunotherapy prolonged survival in a murine GBM model. FIG. 15A, schematic of experimental protocol. FIG. 15B, survival in animals treated as indicated. FIG. 15C, long term survival in animals treated with AAV.CPP16-anti-PDL1. LTS: long-term survival.

FIGS. 16A-C show that GBM tumors were eradicated in all of the long-term surviving mice. FIG. 16A, H&E staining of brain sections both posterior and anterior to the tumor injection site. No residual GBM in any section. FIG. 16B, Bioluminescent imaging 7 days after tumor implant suggesting success of initial tumor implantation. FIG. 16C, GBM tumor implantation site with scar-like tissues.

FIGS. 17A-17B show expression of HA-tagged antiPD-L1 antibody in GBM tumor as measured by Western blotting. AAVs of 1e12 vg or PBS were injected intravenously 5 days after tumor implantation in mice. Tumor tissues were harvested 14 days after IV injection. The intensities of HA tag staining (FIG. 17A) were quantified as measurement of antiPD-L1 antibody expression (FIG. 17B).

### DETAILED DESCRIPTION

Difficulties associated with delivery across the BBB have hindered development of therapeutic agents to treat brain disorders including cancer. Adeno-associated virus (AAV) has emerged as an important research and clinical tool for delivering therapeutic genes to the brain, spinal cord and the eye; see, e.g., U.S. Pat. Nos. 9,102,949; 9,585,971; and US20170166926. Gene therapy mediated by AAVs has made significant progress with the recent approvals of Luxturna and Zolgensma. The approval of Zolgensma for intravascular treatment of spinal muscular atrophy patients under two years of age is particular encouraging, as it demonstrates the feasibility of using BBB-crossing AAV vectors for systemic gene therapy of the central nervous system (CNS). Despite of its success in young patients, AAV9, which is the AAV serotype used in Zolgensma, suffers from low efficiency of BBB crossing, particularly in adults, which limits its application for other CNS diseases^{8,9}. Described herein are next-generation, brain-penetrant AAV vectors (namely, AAV.CPP16) that achieves at least 5-10 fold enhancement over current industrial standard (i.e. AAV9) in both rodents and non-human primates, that can be used for a new BBB-crossing AAV platform for GBM cancer gene therapy.

Through rational design and targeted screening on the basis of known cell-penetrating peptides (CPPs) (see, e.g., Gomez et al., Bax-inhibiting peptides derived from Ku70 and cell-penetrating pentapeptides. Biochem. Soc. Trans. 2007; 35(Pt 4):797-801), targeting sequences have been discovered that, when engineered into the capsid of an AAV, improved the efficiency of gene delivery to the brain by up to three orders of magnitude. These methods were used to engineer AAV vectors that dramatically reduce tumor size in an animal model of glioblastoma.

In addition, the brain is “immune privileged”, which renders immunotherapy of GBM challenging. “Priming” the immune response is desirable to turn the immunologically “cold” GBM tumor into an immunogenic, “hot” one. The present methods make use of the vectors described herein to deliver immunotherapeutics that may achieve just that, e.g., anti-PD-L1 antibodies. Without wishing to be bound by theory, it is believed that the AAV vector itself “primes” the immune system by increasing tumor infiltration of cytotoxic T cells while the antiPD-L1 antibody expressed at the tumor site, and in the CNS at large, activates the otherwise “exhausted” T cells.

### Targeting Sequences

The present methods identified a number of potential targeting peptides that enhance permeation through the BBB, e.g., when inserted into the capsid of an AAV, e.g., AAV1, AAV2, AAV8, or AAV9, or when conjugated to a biological agent, e.g., an antibody or other large biomolecule, either chemically or via expression as a fusion protein.

In some embodiments, the targeting peptides comprise sequences of at least 5 amino acids. In some embodiments, the amino acid sequence comprises at least 4, e.g., 5, contiguous amino acids of the sequences VPALR (SEQ ID NO:1) and VSALK (SEQ ID NO:2).

In some embodiments, the targeting peptides comprise a sequence of X₁ X₂ X₃ X₄ X₅, wherein:
(i) X₁, X₂, X₃, X₄ are any four non-identical amino acids of V, A, L, I, G, P, S, T, or M; and
(ii) X₅ is K, R, H, D, or E (SEQ ID NO:73).

In some embodiments, the targeting peptides comprise sequences of at least 6 amino acids. In some embodiments, the amino acid sequence comprises at least 4, e.g., 5 or 6 contiguous amino acids of the sequences TVPALR (SEQ ID NO:3), TVSALK (SEQ ID NO:4), TVPMLK (SEQ ID NO:12) and TVPTLK (SEQ ID NO:13).

In some embodiments, the targeting peptides comprise a sequence of X₁ X₂ X₃ X₄ X₅ X₆, wherein:
(i) X₁ is T;
(ii) X₂, X₃, X₄, X₅ are any four non-identical amino acids of V, A, L, I, G, P, S, T, or M; and
(iii) X₆ is K, R, H, D, or E (SEQ ID NO:74).

In some embodiments, the targeting peptides comprise a sequence of X₁ X₂ X₃ X₄ X₅ X₆, wherein:
(i) X₁, X₂, X₃, X₄ are any four non-identical amino acids from V, A, L, I, G, P, S, T, or M;
(ii) X₅ is K, R, H, D, or E; and
(iii) X₆ is E or D (SEQ ID NO:75).

In some embodiments, the targeting peptides comprise sequences of at least 7 amino acids. In some embodiments, the amino acid sequence comprises at least 4, e.g., 5, 6, or 7 contiguous amino acids of the sequences FTVSALK (SEQ ID NO:5), LTVSALK (SEQ ID NO:6), TVSALFK (SEQ ID NO:8), TVPALFR (SEQ ID NO:9), TVPMLFK (SEQ ID NO:10) and TVPTLFK (SEQ ID NO:11). In some other embodiments, the targeting peptides comprise a sequence of X₁ X₂ X₃ X₄ X₅ X₆ X₇, wherein:
(i) X₁ is F, L, W, or Y;
(ii) X₂ is T;
(iii) X₃, X₄, X₅, X₆ are any four non-identical amino acids of V, A, L, I, G, P, S, T, or M; and
(iv) X₇ is K, R, H, D, or E (SEQ ID NO:76).

In some embodiments, the targeting peptides comprise a sequence of X₁ X₂ X₃ X₄ X₅ X₆ X₇, wherein:
(i) X₁ is T;
(ii) X₂, X₃, X₄, X₅ are any four non-identical amino acids of V, A, L, I, G, P, S, T, or M;
(iii) X₆ is K, R, H, D, or E; and
(iv) X₇ is E or D (SEQ ID NO:77).

In some embodiments, the targeting peptides comprise a sequence of X₁ X₂ X₃ X₄ X₅ X₆ X₇, wherein:
(i) X₁, X₂, X₃, X₄ are any four non-identical amino acids of V, A, L, I, G, P, S, T, or M;
(ii) X₅ is K, R, H, D, or E;
(iii) X₆ is E or D; and
(iv) X₇ is A or I (SEQ ID NO:78).

In some embodiments, the targeting peptides comprise a sequence of V[S/p][A/m/t/]L (SEQ ID NO:79), wherein the upper case letters are preferred at that position. In some embodiments, the targeting peptides comprise a sequence of TV[S/p][A/m/t/]L (SEQ ID NO:80). In some embodiments, the targeting peptides comprise a sequence of TV[S/p][A/m/t/]LK (SEQ ID NO:81). In some embodiments, the targeting peptides comprise a sequence of TV[S/p][A/m/t/]LFK. (SEQ ID NO:82).

In some embodiments, the targeting peptide does not consist of VPALR (SEQ ID NO:1) or VSALK (SEQ ID NO:2).

Specific exemplary amino acid sequences that include the above mentioned 5, 6, or 7-amino acid sequences are listed in Table 1.

TABLE 1
Targeting Sequences

| SEQ ID NO: | Targeting Peptide Sequence |
|---|---|
| 1. | VPALR |
| 2. | VSALK |
| 3. | TVPALR |
| 4. | TVSALK |
| 5. | FTVSALK |
| 6. | LTVSALK |
| 7. | TFVSALK |
| 8. | TVSALFK |
| 9. | TVPALFR |
| 10. | TVPMLFK |
| 11. | TVPTLFK |
| 12. | TVPMLK |
| 13. | TVPTLK |
| 14. | VPMLK |
| 15. | VPTLK |
| 16. | VPMLKE |
| 17. | VPTLKD |
| 18. | VPALRD |
| 19. | VSALKE |
| 20. | VSALKD |
| 21. | TAVSLK |
| 22. | TALVSK |
| 23. | TVLSAK |
| 24. | TLVSAK |
| 25. | TMVPLK |
| 26. | TMLVPK |
| 27. | TVLPMK |
| 28. | TLVPMK |
| 29. | TTVPLK |
| 30. | TTLVPK |
| 31. | TVLPTK |
| 32. | TLVPTK |
| 33. | TAVPLR |
| 34. | TALVPR |
| 35. | TVLPAR |
| 36. | TLVPAR |
| 37. | TAVSLKE |
| 38. | TALVSKE |
| 39. | TVLSAKE |
| 40. | TLVSAKE |
| 41. | TMVPLKE |
| 42. | TMLVPKE |
| 43. | TVLPMKE |
| 44. | TLVPMKE |
| 45. | TTVPLKD |
| 46. | TTLVPKD |
| 47. | TVLPTKD |
| 48. | TLVPTKD |
| 49. | TAVPLRD |
| 50. | TALVPRD |
| 51. | TVLPARD |
| 52. | TLVPARD |
| 53. | TAVSLFK |
| 54. | TALVSFK |
| 55. | TVLSAFK |
| 56. | TLVSAFK |
| 57. | TMVPLFK |
| 58. | TMLVPFK |
| 59. | TVLPMFK |
| 60. | TLVPMFK |
| 61. | TTVPLFK |
| 62. | TTLVPFK |
| 63. | TVLPTFK |
| 64. | TLVPTFK |
| 65. | TAVPLFR |
| 66. | TALVPFR |
| 67. | TVLPAFR |
| 68. | TLVPAFR |

Targeting peptides including reversed sequences can also be used, e.g., KLASVT (SEQ ID NO:83) and KFLASVT (SEQ ID NO:84).

Targeting peptides disclosed herein can be modified according to the methods known in the art for producing peptidomimetics. See, e.g., Qvit et al., Drug Discov Today. 2017 February; 22(2): 454-462; Farhadi and Hashemian, Drug Des Devel Ther. 2018; 12: 1239-1254; Avan et al., Chem. Soc. Rev., 2014, 43, 3575-3594; Pathak, et al., Indo American Journal of Pharmaceutical Research, 2015. 8; Kazmierski, W. M., ed., Peptidomimetics Protocols, Human Press (Totowa NJ 1998); Goodman et al., eds., Houben-Weyl Methods of Organic Chemistry: Synthesis of Peptides and Peptidomimetics, Thiele Verlag (New York 2003); and Mayo et al., J. Biol. Chem., 278:45746 (2003). In some cases, these modified peptidomimetic versions of the peptides and fragments disclosed herein exhibit enhanced stability in vivo, relative to the non-peptidomimetic peptides.

Methods for creating a peptidomimetic include substituting one or more, e.g., all, of the amino acids in a peptide sequence with D-amino acid enantiomers. Such sequences are referred to herein as “retro” sequences. In another method, the N-terminal to C-terminal order of the amino acid residues is reversed, such that the order of amino acid residues from the N-terminus to the C-terminus of the original peptide becomes the order of amino acid residues from the C-terminus to the N-terminus in the modified peptidomimetic. Such sequences can be referred to as “inverso” sequences.

Peptidomimetics can be both the retro and inverso versions, i.e., the “retro-inverso” version of a peptide disclosed herein. The new peptidomimetics can be composed of D-amino acids arranged so that the order of amino acid residues from the N-terminus to the C-terminus in the peptidomimetic corresponds to the order of amino acid residues from the C-terminus to the N-terminus in the original peptide.

Other methods for making a peptidomimetic include replacing one or more amino acid residues in a peptide with a chemically distinct but recognized functional analog of the amino acid, i.e., an artificial amino acid analog. Artificial amino acid analogs include ϑ-amino acids, ϑ-substituted ϑ-amino acids (“ϑ³-amino acids”), phosphorous analogs of amino acids, such as ∀-amino phosphonic acids and ∀-amino phosphinic acids, and amino acids having non-peptide linkages. Artificial amino acids can be used to create peptidomimetics, such as peptoid oligomers (e.g., peptoid amide or ester analogues), ϑ-peptides, cyclic peptides, oligourea or oligocarbamate peptides; or heterocyclic ring molecules. Exemplary retro-inverso targeting peptidomimetics include KLASVT and KFLASVT, wherein the sequences include all D-amino acids. These sequences can be modified, e.g., by biotinylation of the amino terminus and amidation of the carboxy terminus.

### AAVs

Viral vectors for use in the present methods and compositions include recombinant retroviruses, adenovirus, adeno-associated virus, alphavirus, and lentivirus, comprising the targeting peptides described herein and optionally a transgene for expression in a target tissue.

A preferred viral vector system useful for delivery of nucleic acids in the present methods is the adeno-associated virus (AAV). AAV is a tiny non-enveloped virus having a 25 nm capsid. No disease is known or has been shown to be associated with the wild type virus. AAV has a single-stranded DNA (ssDNA) genome. AAV has been shown to exhibit long-term episomal transgene expression, and AAV has demonstrated excellent transgene expression in the brain, particularly in neurons. Vectors containing as little as 300 base pairs of AAV can be packaged and can integrate. Space for exogenous DNA is limited to about 4.7 kb. An AAV vector such as that described in Tratschin et al., Mol. Cell. Biol. 5:3251-3260 (1985) can be used to introduce DNA into cells. A variety of nucleic acids have been introduced into different cell types using AAV vectors (see for example Hermonat et al., Proc. Natl. Acad. Sci. USA 81:6466-6470 (1984); Tratschin et al., Mol. Cell. Biol. 4:2072-2081 (1985); Wondisford et al., Mol. Endocrinol. 2:32-39 (1988); Tratschin et al., J. Virol. 51:611-619 (1984); and Flotte et al., J. Biol. Chem. 268:3781-3790 (1993). There are numerous alternative AAV variants (over 100 have been cloned), and AAV variants have been identified based on desirable characteristics. In some embodiments, the AAV is AAV1, AAV2, AAV4, AAV5, AAV6, AV6.2, AAV7, AAV8, AAV9, rh.10, rh.39, rh.43 or CSp3; for CNS use, in some embodiments the AAV is AAV1, AAV2, AAV4, AAV5, AAV6, AAV8, or AAV9. As one example, AAV9 has been shown to somewhat efficiently cross the blood-brain barrier. Using the present methods, the AAV capsid can be genetically engineered to increase permeation across the BBB, or into a specific tissue, by insertion of a targeting sequence as described herein into the capsid protein, e.g., into the AAV9 capsid protein VP1 between amino acids 588 and 589.

An exemplary wild type AAV9 capsid protein VP1 (Q6JC40-1) sequence is as follows:

```
(SEQ ID NO: 85)
                10 20 30 40
MAADGYLPDW LEDNLSEGIR EWWALKPGAP QPKANQQHQD
                50 60 70 80
NARGLVLPGY KYLGPGNGLD KGEPVNAADA AALEHDKAYD
                90 100 110 120
QQLKAGDNPY LKYNHADAEF QERLKEDTSF GGNLGRAVFQ
              130 140 150 160
AKKRLLEPLG LVEEAAKTAP GKKRPVEQSP QEPDSSAGIG
              170 180 190 200
KSGAQPAKKR LNFGQTGDTE SVPDPQPIGE PPAAPSGVGS
              210 220 230 240
LTMASGGGAP VADNNEGADG VGSSSGNWHC DSQWLGDRVI
              250 260 270 280
TTSTRTWALP TYNNHLYKQI SNSTSGGSSN DNAYFGYSTP
              290 300 310 320
WGYFDFNRFH CHFSPRDWQR LINNNWGFRP KRLNFKLFNI
              330 340 350 360
QVKEVTDNNG VKTIANNLTS TVQVFTDSDY QLPYVLGSAH
              370 380 390 400
EGCLPPFPAD VFMIPQYGYL TLNDGSQAVG RSSFYCLEYF
              410 420 430 440
PSQMLRTGNN FQFSYEFENV PFHSSYAHSQ SLDRLMNPLI
              450 460 470 480
DQYLYYLSKT INGSGQNQQT LKFSVAGPSN MAVQGRNYIP
              490 500 510 520
GPSYRQQRVS TTVTQNNNSE FAWPGASSWA LNGRNSLMNP
              530 540 550 560
GPAMASHKEG EDRFFPLSGS LIFGKQGTGR DNVDADKVMI
              570 580 590 600
TNEEEIKTTN PVATESYGQV ATNHQSAQAQ AQTGWVQNQG
              610 620 630 640
ILPGMVWQDR DVYLQGPIWA KIPHTDGNFH PSPLMGGFGM
              650 660 670 680
KHPPPQILIK NTPVPADPPT AFNKDKLNSF ITQYSTGQVS
              690 700 710 720
VEIEWELQKE NSKRWNPEIQ YTSNYYKSNN VEFAVNTEGV
              730
YSEPRPIGTR YLTRNL
```

Thus provided herein are AAV that include one or more of the targeting peptide sequences described herein, e.g., an AAV comprising a capsid protein comprising a targeting sequence described herein, e.g., a capsid protein comprising SEQ ID NO:1 wherein a targeting peptide sequence has been inserted into the sequence, e.g., between amino acids 588 and 589.

### Immunotherapeutic Transgenes

In some embodiments, the AAV also includes a transgene sequence (i.e., a heterologous sequence) encoding an immunotherapeutic agent, e.g., as described herein or as known in the art. The transgene is preferably linked to sequences that promote/drive expression of the transgene in the target tissue.

Exemplary transgenes for use as immunotherapeutics include those encoding an immune checkpoint inhibitory antibody or antigen-binding fragment thereof, e.g., single-chain variable fragment (scFv) antibodies that act as checkpoint inhibitors.

Examples of immunotherapies include, but are not limited to, adoptive T cell therapies or cancer vaccine preparations designed to induce T lymphocytes to recognize cancer cells, as well as checkpoint inhibitors such as anti-CD137 antibodies (e.g., BMS-663513), anti-PD1 antibodies (e.g., Nivolumab, pembrolizumab/MK-3475, Pidilizumab (CT-011)), anti-PDL1 antibodies (e.g., BMS-936559, MPDL3280A), or anti-CTLA-4 antibodies (e.g., ipilumimab; see, e.g., Kruger et al. (2007) Histol Histopathol. 22(6): 687-96; Eggermont et al. (2010) Semin Oncol. 37(5): 455-9; Klinke (2010) Mol. Cancer. 9: 242; Alexandrescu et al. (2010) J. Immunother. 33(6): 570-90; Moschella et al. (2010) Ann NY Acad Sci. 1194: 169-78; Ganesan and Bakhshi (2010) Natl. Med. J. India 23(1): 21-7; and Golovina and Vonderheide (2010) Cancer J. 16(4): 342-7.

Exemplary anti-PD-1 antibodies that can be used in the methods described herein include those that bind to human PD-1; an exemplary PD-1 protein sequence is provided at NCBI Accession No. NP_005009.2. Exemplary antibodies are described in U.S. Pat. Nos. 8,008,449; 9,073,994; and U.S. Publication No. 2011/0271358, including, e.g., PF-06801591, AMP-224, BGB-A317, BI 754091, JS001, MEDI0680, PDR001, REGN2810, SHR-1210, TSR-042, pembrolizumab, nivolumab, avelumab, Cemiplimab, Spartalizumab, Camrelizumab, Sintilimab, pidilizumab, Tislelizumab, Toripalimab, AMP-224, AMP-514, and atezolizumab.

Exemplary anti-CD40 antibodies that can be used in the methods described herein include those that bind to human CD40; exemplary CD40 protein precursor sequences are provided at NCBI Accession No. NP_001241.1, NP_690593.1, NP_001309351.1, NP_001309350.1 and NP_001289682.1. Exemplary antibodies include those described in International Publication Nos. WO 2002/088186; WO 2007/124299; WO 2011/123489; WO 2012/149356; WO 2012/111762; WO 2014/070934; U.S. Publication Nos. 2013/0011405; 2007/0148163; 2004/0120948; 2003/0165499; and U.S. Pat. No. 8,591,900; including, e.g., dacetuzumab, lucatumumab, bleselumab, teneliximab, ADC-1013, CP-870,893, Chi Lob 7/4, HCD122, SGN-4, SEA-CD40, BMS-986004, and APX005M. In some embodiments, the anti-CD40 antibody is a CD40 agonist, and not a CD40 antagonist.

Exemplary anti-PD-L1 antibodies that can be used in the methods described herein include those that bind to human PD-L1; exemplary PD-L1 protein sequences are provided at NCBI Accession No. NP_001254635.1, NP_001300958.1, and NP_054862.1. Exemplary antibodies are described in U.S. Publication No. 2017/0058033; International Publication Nos. WO 2017/118321A1; WO 2016/061142A1; WO 2016/007235A1; WO 2014/195852A1; and WO 2013/079174A1, including, e.g., BMS-936559 (MDX-1105), FAZ053, KN035, Atezolizumab (Tecentriq, MPDL3280A), Avelumab (Bavencio), Durvalumab (Imfinzi, MEDI-4736), Envafolimab (KN035), CK-301, CS-1001, SHR-1316 (HTI-1088), CBT-502 (TQB-2450), BGB-A333, and BMS-986189. Non antibody peptide inhibitors can also be used, e.g., AUNP12, CA-170. See also Akinleye & Rasool, Journal of Hematology & Oncology 12:92 (2019) doi:10.1186/si3045-019-0779-5.

In some embodiments, the immunotherapeutic is or comprises an antigen binding portion of anti-PD-L1 antibody, e.g., single-chain variable fragment (scFv) antibodies against human PD-L1 protein (PD-L1.Hu); an exemplary sequence encoding an anti-PDL1 antibody scFv is shown in SEQ ID NO:105, or a portion thereof, e.g., lacking one, two or more of the signal peptide, HA-tag, and Myc-tag, e.g., comprising amino acids (aa) 31-513 of SEQ ID NO:105:

Exemplary anti-PDL1 scFv sequence (Signal peptide (aa 1-21); HA-tag, aa
21-30; Myc-tag, aa 514-523)
(SEQ ID NO: 105)
METDTLLLWVLLLWVPGSTGDYPYDVPDYAGAQPADDIQMTQSPSSLSASV
GDRVTITCRASQDVSTAVAWYQQKPGKAPKLLIYSASFLYSGVPSRFSGSGSG
TDFTLTISSLQPEDFATYYCQQYLYHPATFGQGTKVEIKRGGGGSGGGGSGG
GGSEVOLVESGGGLVQPGGSLRLSCAASGFTFSDSWIHWVRQAPGKGLEWV
AWISPYGGSTYYADSVKGRFTISADTSKNTAYLQMNSLRAEDTAVYYCARR
HWPGGFDYWGQGTLVTVSAVDEAKSCDKTHTCPPCPAPELLGGPSVFLFPPK
PKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLDSDG

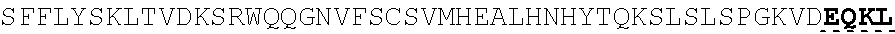

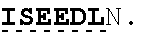

The following is an exemplary antiPD-L1 nucleic acid sequence (Signal

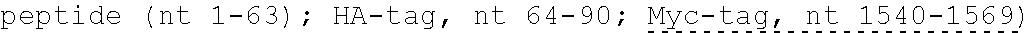

(SEQ ID NO: 106)
ATGGAGACAGACACACTCCTGCTATGGGTACTGCTGCTCTGGGTTCC
AGGTTCCACTGGTGACTATCCATATGATGTTCCAGATTATGCTGGGGCCCAG
CCGGCCGACGACATCCAAATGACCCAGAGTCCATCTAGTCTGTCTGCTTC
GGTAGGTGATAGGGTCACTATTACTTGCAGGGCCTCCCAGGACGTGTCAA
CTGCAGTGGCTTGGTACCAACAGAAGCCCGGGAAAGCTCCCAAACTGCTG
ATCTACTCCGCCAGCTTTCTGTATTCCGGAGTTCCGTCTAGATTTTCCGGA
TCAGGAAGCGGCACGGATTTCACACTCACAATAAGCAGCCTACAACCAGA
GGACTTCGCAACCTACTATTGTCAACAGTACCTGTACCATCCAGCCACCTT
TGGGCAGGGCACCAAGGTGGAAATCAAGCGCGGTGGTGGTGGATCAGGT
GGAGGCGGAAGTGGAGGTGGCGGATCCGAAGTTCAGCTTGTCGAGTCCG
GTGGCGGCCTGGTTCAGCCTGGTGGGTCTTTGCGTCTGTCATGCGCCGCCT
CTGGTTTCACCTTTTCAGACTCTTGGATCCACTGGGTGAGACAGGCCCCAG
GAAAGGGTCTTGAGTGGGTTGCATGGATTAGCCCCTACGGCGGCAGTACA
TATTACGCGGATAGCGTGAAAGGGAGGTTTACCATCAGCGCAGACACGTC
GAAGAACACCGCATACCTCCAGATGAATTCCCTCCGAGCCGAAGATACCG
CCGTGTACTATTGTGCTCGCCGGCATTGGCCTGGCGGCTTCGATTATTGGG
GACAGGGAACTCTAGTAACAGTGTCGGCTGTCGACGAGGCCAAATCTTGT
GACAAAACTCACACATGCCCACCGTGCCCAGCACCCGAACTCCTGGGGGG
ACCGTCAGTCTTCCTCTTCCCCCCAAAACCCAAGGACACCCTCATGATCTC
CCGGACCCCTGAGGTCACATGCGTGGTGGTGGACGTGAGCCACGAAGACC
CTGAGGTCAAGTTCAACTGGTACGTGGACGGCGTGGAGGTGCATAATGCC
AAGACAAAGCCGCGGGAGGAGCAGTACAACAGCACGTACCGTGTGGTCA
GCGTCCTCACCGTCCTGCACCAGGACTGGCTGAATGGCAAGGAGTACAAG
TGCAAGGTCTCCAACAAAGCCCTCCCAGCCCCCATCGAGAAAACCATCTC
CAAAGCCAAGGGGCAGCCCCGAGAACCACAGGTGTACACCCTGCCCCCAT
CCCGGGATGAGCTGACCAAGAACCAGGTCAGCCTGACCTGCCTGGTCAAA
GGCTTCTATCCCAGCGACATCGCCGTGGAGTGGGAGAGCAATGGGCAGCC
GGAGAACAACTACAAGACCACGCCTCCCGTGCTGGACTCCGACGGCTCCT
TCTTCCTCTACAGCAAGCTCACCGTGGACAAGAGCAGGTGGCAGCAGGGG
AACGTCTTCTCATGCTCCGTGATGCATGAGGCTCTGCACAACCACTACACG

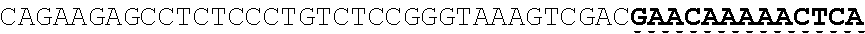

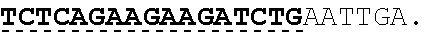

Other antibodies, as well as methods for generating a nucleic acid encoding such antibodies are known in the art; see, e.g., Li et al., Int J Mol Sci. 2016 July; 17(7): 1151; Engeland et al., Mol Ther. 2014 November; 22(11): 1949-1959 and the references above.

The virus can also include one or more sequences that promote expression of a transgene, e.g., one or more promoter sequences; enhancer sequences, e.g., 5′ untranslated region (UTR) or a 3′ UTR; a polyadenylation site; and/or insulator sequences. In some embodiments, the promoter is a brain tissue specific promoter, e.g., a neuron-specific or glia-specific promoter. In certain embodiments, the promoter is a promoter of a gene selected to from: neuronal nuclei (NeuN), glial fibrillary acidic protein (GFAP), MeCP2, adenomatous polyposis coli (APC), ionized calcium-binding adapter molecule 1 (Iba-1), synapsin I (SYN), calcium/calmodulin-dependent protein kinase II, tubulin alpha I, neuron-specific enolase and platelet-derived growth factor beta chain. In some embodiments, the promoter is a pan-cell type promoter, e.g., cytomegalovirus (CMV), beta glucuronidase, (GUSB), ubiquitin C (UBC), or rous sarcoma virus (RSV) promoter. The woodchuck hepatitis virus posttranscriptional response element (WPRE) can also be used. In some embodiments, a human signal or leader sequence, e.g., an IgK leader sequence is used. In some embodiments, a human signal sequence is used instead, as shown in the following table (Table adapted from novoprolabs.com/support/articles/commonly-used-leader-peptide-sequences-for-efficient-secretion-of-a-recombinant-protein-expressed-in-mammalian-cells-201804211337.html):

| Human | | SEQ |
|---|---|---|
| Signal | | ID |
| sequence | Sequence | NO: |
| Oncostatin M | MGVLLTQRTLLSLVLAL | 107 |
| | LFPSMASM | |
| IgG2 H | MGWSCIILFLVATATGVHS | 108 |
| Secrecon | MWWRLWWLLLLLLLLWPMVWA | 109* |
| IgKVIII | MDMRVPAQLLGLLLLWLRGARC | 110 |
| CD33 | MPLLLLLPLLWAGALA | 111 |
| tPA | MDAMKRGLCCVLLLCGA | 112 |
| | VFVSPS | |
| Human | MAFLWLLSCWALLGTTFG | 113 |
| Chymotrypsinogen | | |
| Human | MNLLLILTFVAAAVA | 114 |
| trypsinogen-2 | | |
| Human IL-2 | MYRMQLLSCIALSLALVTNS | 115 |
| Albumin (HSA) | MKWVTFISLLFSSAYS | 116 |
| Human insulin | MALWMRLLPLLALLALW | 117 |
| | GPDPAAA | |

*Barash et al., Biochem Biophys Res Commun. 2002 Jun. 21; 294(4): 835-42.

In some embodiments, a secretory sequence that promotes secretion of the antibody is used, e.g., as described in von Heijne, J Mol Biol. 1985 Jul 5;184(1):99-105; Kober et al., Biotechnol. Bioeng. 2013; 110: 1164-1173; Tsuchiya et al., Nucleic Acids Research Supplenzent No. 3 261-262 (2003).

In some embodiments, the AAV also has one or more additional mutations that increase delivery to the target tissue, e.g., the CNS, or that reduce off-tissue targeting, e.g., mutations that decrease liver delivery when CNS, heart, or muscle delivery is intended (e.g., as described in Pulicherla et al. (2011) Mol Ther 19:1070-1078); or the addition of other targeting peptides, e.g., as described in Chen et al. (2008) Nat Med 15:1215-1218 or Xu et al., (2005) Virology 341:203-214 or U.S. Pat. Nos. 9,102,949; 9,585,971; and US20170166926. See also Gray and Samulski (2011) “Vector design and considerations for CNS applications,” in Gene Vector Design and Application to Treat Nervous System Disorders ed. Glorioso J., editor. (Washington, DC: Society for Neuroscience) 1-9, available at sfn.org/˜/media/SfN/Documents/Short %20Courses/2011%20Short %20Course %20I/2 011_SC1_Gray.ashx.

### Methods of Use

The methods and compositions described herein can be used to deliver an immunotherapeutic composition to a tissue, e.g., to the central nervous system (brain), heart, muscle, or dorsal root ganglion or spinal cord (peripheral nervous system). In some embodiments, the methods include delivery to specific brain regions, e.g., cortex, cerebellum, hippocampus, substantia nigra, or amygdala. In some embodiments, the methods include delivery to neurons, astrocytes, and/or glial cells.

In some embodiments, the methods and compositions, e.g., AAVs, are used to deliver a nucleic acid sequence encoding an immunotherapeutic to a subject who has brain cancer. Brain cancers include gliomas (e.g., glioblastoma multiforme (GBM)), metastases (e.g., from lung, breast, melanoma, or colon cancer), meningiomas, pituitary adenomas, and acoustic neuromas. Thus the methods can include systemically, e.g., intravenously, administering an AAV (e.g., AAV9) comprising a targeting peptide as described herein (e.g., AAV9 with a CPP 16 inserted therein, also referred to herein as AAV.CPP16) and encoding an immunotherapeutic to a subject who has been diagnosed with brain cancer.

In some embodiments, the methods also include co-administering a chemotherapeutic agent. In some embodiments, the chemotherapeutic agent is a toxin or cytotoxic drug, including but not limited to temozolamide, lomustine, or a combination thereof. See, e.g., Herrlinger et al., Lancet. 2019 Feb. 16; 393(10172):678-688. The methods can also include administering radiation, surgical resection, or both.

### Pharmaceutical Compositions and Methods of Administration

The methods described herein include the use of pharmaceutical compositions comprising AAVs comprising (i) the targeting peptides and (ii) sequences encoding an immunotherapeutic as an active ingredient.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration.

Pharmaceutical compositions are typically formulated to be compatible with its intended route of administration. Examples of routes of administration include parenteral, e.g., intravenous, intraarterial, subcutaneous, intraperitoneal intramuscular or injection or infusion administration. Delivery can thus be systemic or localized.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a kit, container, pack, or dispenser together with instructions for administration.

### EXAMPLES

The invention is further described in the following examples, which do not limit the scope of the invention described in the claims.

### Materials and Methods

The following materials and methods were used in the Examples below.

### 1. Generation of Capsid Variants

To generate the capsid variant plasmids, DNA fragments that encode the cell-penetrating peptides (Table 3) were synthesized (GenScript), and inserted into the backbone of the AAV9 Rep-cap plasmid (pRC9) between amino acid position 588 and 589 (VP1 amino acid numbering), using CloneEZ seamless cloning technology (GenScript). CPPs BIP1 (VPALR, SEQ ID NO:1) and BIP2 (VSALK, SEQ ID NO:2), as well as their derivatives such as TVSALK (SEQ ID NO:4) in AAV.CPP.16 and TVSALFK (SEQ ID NO:8) in AAV.CPP.21, are derived from the Ku70 proteins, of which the sequences are provided as below:

Human Ku70 MSGWESYYKTEGDEEAEEEQEENLEASGDYKYSGRDSLIFLVDASKAMFESQSEDELTPE 60
Mouse Ku70 MSEWESYYKTEGEEEEEE--EESPDTGGEYKYSGRDSLIFLVDASRAMFESQGEDELTPF 58
Rat Ku70 MSEWESYYKTEGEEEEEE--EQSPDTNGEYKYSGRDSLIFLVDASRAMFESQGEDELTPF 58
Human Ku70 DMSIQCIQSVYISKIISSDRDLLAVVFYGTEKDKNSVNFKNIYVLQELDNPGAKRILELD 120
Mouse Ku70 DMSIQCIQSVYTSKIISSDRDLLAVVFYGTEKDKNSVNFKNIYVLQDLDNPGAKRVLELD 118
Rat Ku70 DMSIQCIQSVYTSKIISSDRDLLAVVFYGTEKDKNSVNFKSIYVLQDLDNPGAKRVLELD 118
Human Ku70 QFKGQQGQKRFQDMMGHGSDYSLSEVLWVCANLFSDVQFKMSHKRIMLFTNEDNPHGNDS 180
Mouse Ku70 QFKGQQGKKHFRDTVGHGSDYSLSEVLWVCANLFSDVQLKMSHKRIMLFTNEDDPHGRDS 178
Rat Ku70 RFKGQQGKKHFRDTIGHGSDYSLSEVLWVCANLFSDVQFKMSHKRIMLFTNEDDPHGNDS 178
Human Ku70 AKASRARTKAGDLRDTGIFLDLMHLKKPGGFDISLFYRDIISIAEDEDLRVHFEESSKLE 240
Mouse Ku70 AKASRARTKASDLRDTGIFLDLMHLKKPGGFDVSVFYRDIITTAEDEDLGVHFEESSKLE 238
Rat Ku70 AKASRARTKASDLRDTGIFLDLMHLKKRGGFDVSLFYRDIISIAEDEDLGVHFEESSKLE 238
Human Ku70 DLLRKVRAKETRKRALSRLKLKLNKDIVISVGIYNLVQKALKPPPIKLYRETNEPVKTKT 300
Mouse Ku70 DLLRKVRAKETKKRVLSRLKFKLGEDVVLMVGIYNLVQKANKPFPVRLYRETNEPVKTKT 298
Rat Ku70 DLLRKVRAKETKKRVLSRLKFKLGKDVALMVGVYNLVQKANKPFPVRLYRETNEPVKTKT 298
Human Ku70 RTFNTSTGGLLLPSDTKRSQIYGSRQIILEKEETEELKRFDDPGLMLMGFKPLVLLKKHH 360
Mouse Ku70 RTFNVNTGSLLLPSDTKRSLTYGTRQIVLEKEETEELKRFDEPGLILMGFKPTVMLKKQH 358
Rat Ku70 RTFNVNTGSLLLPSDTKRSLTFGTRQIVLEKEETEELKRFDEPGLILMGFKPMVMLKNHH 358
Human Ku70 YLRPSLFVYPEESLVIGSSTLFSALLIKCLEKEVAALCRYTPRRNIPPYFVALVPQEEEL 420
Mouse Ku70 YLRPSLFVYPEESLVSGSSTLFSALLTKCVEKEVIAVCRYTPRKNVSPYFVALVPQEEEL 418
Rat Ku70 YLRPSLFLYPEESLVNGSSTLFSALLTKCVEKEVIAVCRYTARKNVSPYFVALVPQEEEL 418
Human Ku70 DDQKIQVTPPGFQLVFLPFADDKRKMPFTEKIMATPEQVGKMKAIVEKLRFTYRSDSFEN 480
Mouse Ku70 DDQNIQVTPGGFQLVFLPYADDKRKVPFTEKVTANQEQIDKMKAIVQKLRFTYRSDSFEN 478
Rat Ku70 DDQNIQVTPAGFQLVFLPYADDKRKVPFTEKVMANPEQIDKMKAIVQKLRFTYRSDSFEN 478
Human Ku70 PVLQQHFRNLEALALDLMEPEQAVDLTLPKVEAMNKRLGSLVDEFKELVYPPDYNPEGKV 540
Mouse Ku70 PVLQQHFRNLEALALDMMESEQVVDLTLPKVEAIKKRLGSLADEFKELVYPPGYNPEGKV 538
Rat Ku70 PVLQQHFRNLEALALDMMESEQVVDLTLPKVEAIKKRLGSLADEFKELVYPPGYNPEGKI 538
Human Ku70 TKRKHDNEGSGSKRPKVEYSEEELKTHISKGTLGKFTVPMLKEACRAYGLKSGLKKQELL 600
Mouse Ku70 AKRKQDDEGSTSKKPKVELSEEELKAHFRKGTLGKLTVPTLKDICKAHGLKSGPKKQELL 598
Rat Ku70 AKRKADNEGSASKKPKVELSEEELKDLFAKGTLGKLTVPALRDICKAYGLKSGPKKQELL 598
(SEQ ID NO: 86)
Human Ku70 EALTKHFQD- 609
(SEQ ID NO: 87)
Mouse Ku70 DALIRHLEKN 608
(SEQ ID NO: 88)
Rat Ku70 EALSRHLEKN 608

In addition, VP1 protein sequences for AAV9, AAV.CPP.16 and AAV.CPP.21 are provided as below:

AAV9 MAADGYLPDWLEDNLSEGIREWWALKPGAPQPKANQQHQDNARGLVLPGYKYLGPGNGLD 60
AAV.CPP16 MAADGYLPDWLEDNLSEGIREWWALKPGAPQPKANQQHQDNARGLVLPGYKYLGPGNGLD 60
AAV.CPP21 MAADGYLPDWLEDNLSEGIREWWALKPGAPQPKANQQHQDNARGLVLPGYKYLGPGNGLD 60
AAV9 KGEPVNAADAAALEHDKAYDQQLKAGDNPYLKYNHADAEFQERLKEDTSFGGNLGRAVFQ 120
AAV.CPP16 KGEPVNAADAAALEHDKAYDQQLKAGDNPYLKYNHADAEFQERLKEDTSFGGNLGRAVFQ 120
AAV.CPP21 KGEPVNAADAAALEHDKAYDQQLKAGDNPYLKYNHADAEFQERLKEDTSFGGNLGRAVFQ 120
AAV9 AKKRLLEPLGLVEEAAKTAPGKKRPVEQSPQEPDSSAGIGKSGAQPAKKRLNFGQTGDTE 180
AAV.CPP16 AKKRLLEPLGLVEEAAKTAPGKKRPVEQSPQEPDSSAGIGKSGAQPAKKRLNFGQTGDTE 180
AAV.CPP21 AKKRLLEPLGLVEEAAKTAPGKKRPVEQSPQEPDSSAGIGKSGAQPAKKRLNFGQTGDTE 180
AAV9 SVPDPQPIGEPPAAPSGVGSLTMASGGGAPVADNNEGADGVGSSSGNWHCDSQWLGDRVI 240
AAV.CPP16 SVPDPQPIGEPPAAPSGVGSLTMASGGGAPVADNNEGADGVGSSSGNWHCDSQWLGDRVI 240
AAV.CPP21 SVPDPQPIGEPPAAPSGVGSLTMASGGGAPVADNNEGADGVGSSSGNWHCDSQWLGDRVI 240
AAV9 TTSTRTWALPTYNNHLYKQISNSTSGGSSNDNAYFGYSTPWGYFDFNRFHCHFSPRDWQR 300
AAV.CPP16 TTSTRTWALPTYNNHLYKQISNSTSGGSSNDNAYFGYSTPWGYFDFNRFHCHFSPRDWQR 300
AAV.CPP21 TTSTRTWALPTYNNHLYKQISNSTSGGSSNDNAYFGYSTPWGYFDFNRFHCHFSPRDWQR 300
AAV9 LINNNWGFRPKRLNFKLFNIQVKEVTDNNGVKTIANNLTSTVQVFTDSDYQLPYVLGSAH 360
AAV.CPP16 LINNNWGFRPKRLNFKLFNIQVKEVTDNNGVKTIANNLTSTVQVFTDSDYQLPYVLGSAH 360
AAV.CPP21 LINNNWGFRPKRLNFKLFNIQVKEVTDNNGVKTIANNLTSTVQVFTDSDYQLPYVLGSAH 360
AAV9 EGCLPPFPADVFMIPQYGYLTLNDGSQAVGRSSFYCLEYFPSQMLRTGNNFQFSYEFENV 420
AAV.CPP16 EGCLPPFPADVFMIPQYGYLTLNDGSQAVGRSSFYCLEYFPSQMLRTGNNFQFSYEFENV 420
AAV.CPP21 EGCLPPFPADVFMIPQYGYLTLNDGSOAVGRSSFYCLEYFPSOMLRTGNNFQPSYEFENV 420
AAV9 PFHSSYAHSQSLDRLMNPLIDQYLYYLSKTINGSGQNQQTLKFSVAGPSNMAVQGRNYIP 480
AAV.CPP16 PFHSSYAHSQSLDRLMNPLIDQYLYYLSKTINGSGQNQQTLKFSVAGPSNMAVQGRNYIP 480
AAV.CPP21 PFHSSYAHSQSLDRLMNPLIDQYLYYLSKTINGSGQNQQTLKFSVAGPSNMAVQGRNYIP 480
AAV9 GPSYRQQRVSTTVTQNNNSEFAWPGASSWALNGRNSLMNPGPAMASHKEGEDRFFPLSGS 540
AAV.CPP16 GPSYRQQRVSTTVTQNNNSEFAWPGASSWALNGRNSLMNPGPAMASHKEGEDRFFPLSGS 540
AAV.CPP21 GPSYRQQRVSTTVTONNNSEFAWPGASSWALNGRNSLMNPGPAMASHKEGEDRFFPLSGS 540
AAV9 LIFGKQGTGRDNVDADKVMITNEEEIKTTNPVATESYGQVATNHQSAQ-------AQAQT 593
AAV.CPP16 LIFGKQGTGRDNVDADKVMITNEEEIKTTNPVATESYGQVATNHQSAQTVSAL-KAQAQT 599
AAV.CPP21 LIFGKQGTGRDNVDADKVMITNEEEIKTTNPVATESYGQVATNHQSAQTVSALFKAQAQT 600
AAV9 GWVQNQGILPGMVWQDRDVYLQGPIWAKIPHTDGNFHPSPLMGGFGMKHPPPQILIKNTP 653
AAV.CPP16 GWVQNQGILPGMVWQDRDVYLQGPIWAKIPHTDGNFHPSPLMGGFGMKHPPPQILIKNTP 659
AAV.CPP21 GWVQNQGILPGMVWQDRDVYLQGPIWAKIPHTDGNFHPSPLMGGFGMKHPPPQILIKNTP 660
AAV9 VPADPPTAFNKDKLNSFITQYSTGQVSVEIEWELQKENSKRWNPEIQYTSNYYKSNNVEF 713
AAV.CPP16 VPADPPTAFNKDKLNSFITQYSTGQVSVEIEWELQKENSKRWNPEIQYTSNYYKSNNVEF 719
AAV.CPP21 VPADPPTAFNKDKLNSFITQYSTGQVSVEIEWELQKENSKRWNPEIQYTSNYYKSNNVEF 720
(SEQ ID NO: 85)
AAV9 AVNTEGVYSEPRPIGTRYLTRNL 736
(SEQ ID NO: 89)
AAV.CPP16 AVNTEGVYSEPRPIGTRYLTRNL 742
(SEQ ID NO: 90)
AAV.CPP21 AVNTEGVYSEPRPIGTRYLTRNL 743

### 2. Recombinant AAV Production

Recombinant AAVs were packaged using standard three-plasmid co-transfection protocol (pRC plasmid, pHelper plasmid and pAAV plasmid). pRC9 (or its variant), pHelper and pAAV carrying a transgene (e.g. nucleus-directed RFP H2B-mCherry driven by an ubiquitous EF1a promoter) were co-transfected into HEK 293T cells using polyethylenimine (PEI, Polysciences). rAAVs vectors were collected from serum-free medium 72h and 120h post transfection and from cell at 120h post transfection. AAV particles in the medium were concentrated using a PEG-precipitation method with 8% PEG-8000 (wt/vol). Cell pellets containing viral particles were resuspended and lysed through sonication. Combined viral vectors from PEG-precipitation and cell lysates were treated with DNase and RNase at 37° C. for 30 mins and then purified by iodixanol gradient (15%, 25%, 40% and 60%) with ultracentrifugation (VTi 50 rotor, 40,000 r.p.m, 18° C., 1 h). rAAVs were then concentrated using Millipore Amicon filter unit (UFC910008, 100K MWCO) and formulated in Dulbecco's phosphate buffered saline (PBS) containing 0.001% Pluronic F68 (Gibco).

### 3. AAV Titering

Virus titer was determined by measuring DNase-resistant genome copies using quantitative PCR. pAAV-CAG-GFP was digested with PVUII(NEB) to generate free ends for the plasmid ITRs, and was used for generating a standard curve. Virus samples were incubated with DNase I to eliminate contaminating DNA, followed by sodium hydroxide treatment to dissolve the viral capsid and to release the viral genome. Quantitative PCR was performed using an ITR Forward primer 5′-GGAACCCCTAGTGATGGAGTT (SEQ ID NO:91) and an ITR Reverse primer 5′-CGGCCTCAGTGAGCGA (SEQ ID NO:92). Vector titers were normalized to the rAAV-2 reference standard materials (RSMs, ATCC, cat No:VR-1616, Manassas, VA).

### 4. Administration of AAV in Mice

For intravenous administration, AAV diluted in sterile saline (0.2 ml) was administered through tail vein injection in adult mice (over 6 weeks of age). Animals then survived for three weeks before being euthanized for tissue harvesting. For intracerebral injection, AAV diluted in PBS (10 ul) was injected using a Hamilton syringe with coordinates from bregma: 1.0 mm right, 0.3 backward, 2.6 mm deep. All animal studies were performed in an AAALAC-accredited facility with IACUC approval.

### 5. Mouse Tissue Processing

Anesthetized animals were transcardially perfused with cold phosphate buffered saline (PBS) followed by 4% paraformaldehyde (PFA). Tissues were post-fixed in 4% PFA overnight, and then immersed in 30% sucrose solutions for two days prior to embedding and snap-freezing in OCT. Typically, 80 μm thick brain sections were cut for imaging of native fluorescence, 40 μm thick brain sections for IHC.

### 6. In Vitro Human BBB Spheroid Model

Hot 1% agarose (w/v, 50 ul) was added in a 96-well plate to cool/solidify. Primary human astrocytes (Lonza Bioscience), human brain microvascular pericytes (HBVP, ScienCell Research Laboratories) and human cerebral microvascular endothelial cells (hCMEC/D3; Cedarlane) were then seeded onto the agarose gel in a 1:1:1 ratio (1500 cells of each type). Cells were cultured at 37° C. in a 5% CO2 incubator for 48-72 hours to allow for spontaneous assembly of multicellular BBB spheroids. A multicellular barrier was reported to form at the periphery of the spheroid, mimicking the BBB. AAVs-H2B-mCherry were added to the culture medium, and 4 days later all spheroids were fixed using 4% PFA, transferred into a Nunc Lab-Tek II thin-glass 8-well chambered coverglass (Thermo Scientific), and imaged using a Zeiss LSM710 confocal microscope. The intensity of RFP signal inside the spheroids was examined and used as a “read-out”.

### 7. AAV Administration in Non-Human Primate (NHP)

All NHP studies were performed by a CRO in an AAALAC-accredited facility with IACUC approval. Cynomolgus monkeys were pre-screened for little or no pre-existing neutralizing antibody against AAV9 (titer of <1:5). AAV diluted in PBS/0.001% F68 was injected intravenously (via cephalic vein or femoral vein) using a peristaltic pump. 3 weeks later, animals were subject to transcardial perfusion with PBS, followed by 4% PFA. Tissues were then collected and processed for paraffin embedding and sectioning.

### 8. Immunohistochemistry

Floating staining was performed for mouse tissue sections with primary antibodies diluted in PBS containing 10% donkey serum and 2% Triton X-100. Primary antibodies used include: chicken anti-GFP (1:1000); rabbit anti-RFP (1:1000); mouse anti-NeuN (1:500); rat anti-GFAP (1:500); Goat anti-GFAP (1:500); mouse anti-CD31 (1:500). Secondary antibodies conjugated to fluorophores of Alexa Fluor 488, Alexa Fluor 555 or Alexa Fluor 647 were applied against the primary antibody's host species at a dilution of 1:200.

For paraffin sections of NHP tissue, DAB staining was performed to visualize cells transduced by AAV-AADC. Rabbit anti-AADC antibody (1:500, Millipore) was used as primary antibody.

### 9. AAV Binding Assay

HEK293T cells were cultured at 37° C. in a 5% CO2 incubator. One day after seeding of HEK293T cells in a 24-well plate at a density of 250,000 cells per well, a cDNA plasmid of LY6A was transiently transfected into the cells using a transfection mixture of 200 ul DMEM (31053028; Gibco), 1 ug DNA plasmid and 3 ug of PEI. 48 hours post transfection, cells were placed on ice to chill down for 10 mins. The medium was then changed with 500 ul ice-cold serum-free DMEM medium containing rAAVs-mCherry at MOI of 10000. After incubating on ice for one hour, cells with presumably AAVs bound to their surface were washed with cold PBS for three times and were then subject to genomic DNA isolation. Cell-binding viral particles were quantified by using qPCR with primers specific to mCherry and normalized to HEK293T genomes using human GCG as reference.

### 10. Mouse Model of Glioblastoma

All experiments were performed in compliance with protocols approved by the Animal Care and Use Committees (IACUC) at the Brigham and Women's Hospital and Harvard Medical School. Syngeneic immuno-competent C57BL/6 female mice weighing 20+/−1 g (Envigo) were used. GL261-Luc (100,000 mouse glioblastoma cells) resuspended in 2 L phosphate buffered saline (PBS) was injected intracranially using 10 μl syringe with a 26-gauge needle (80075; Hamilton). A stereotactic frame was used to locate the implantation site (coordinates from bregma in mm: 2 right, 0.5 forward, at a depth of 3.5 into cortex). 7 days later, 200 ul AAV-HSV-TK1 (1E+12 viral genomes, IV) was administered once and ganciclovir (50 mg/kg) was administered daily for 10 days.

### Example 1. Modification of AAV9 Capsid

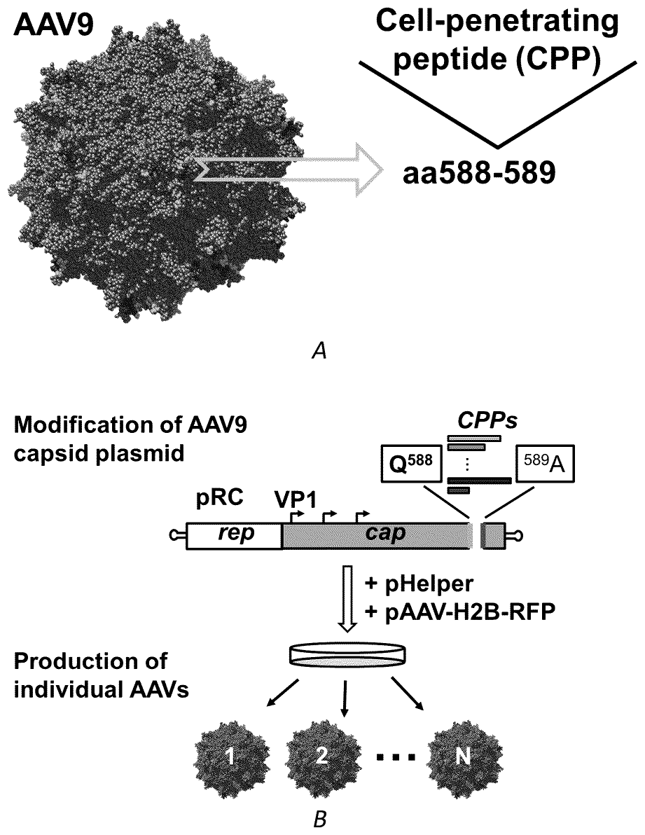

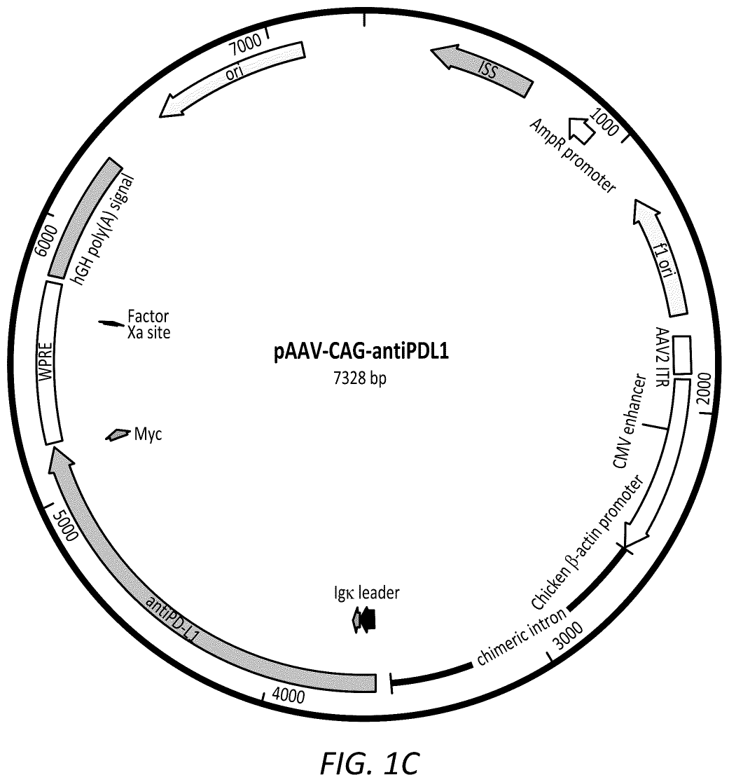

To identify peptide sequences that would enhance permeation of a biomolecule or virus across the blood brain barrier an AAV peptide display technique was used, individual cell-penetrating peptides, as listed in Table 3, were inserted into the AAV9 capsid between amino acids 588 and 589 (VP1 numbering) as illustrated in FIG. 1A. The insertion was carried out by modifying the RC plasmid, one of the three plasmids co-transfected for AAV packaging; FIG. 1B shows an exemplary schematic of the experiments. Individual AAV variants were produced and screened separately. See Materials and Methods #1-3 for more details.

TABLE 3

| | | Name | | | | |
|---|---|---|---|---|---|---|
| | | of | | | No. of | |
| | | CPP | Amino acid | | CPP | Viral |
| | AAV | insert | sequence of CPP | # | residues | titer |
| Initial | AAV9 | N/A | N/A | | N/A | Normal |
| Screening | AAV.CPP.1 | SynB1 | RGGRLSYSRRRFSTSTGR | 93 | 18 | Low |
| | AAV.CPP.2 | L-2 | HARIKPTFRRLKWKY | 94 | 20 | Low |
| | | | KGKFW | | | |
| | AAV.CPP.3 | PreS2-TLM | PLSSIFSRIGDP | 95 | 12 | Low |
| | AAV.CPP.4 | Transportan | AGYLLGKINLKALAA | 96 | 21 | Low |
| | | 10 | LAKKIL | | | |
| | AAV.CPP.5 | SAP | VRLPPPVRLPPPVRLPPP | 97 | 18 | Normal |
| | AAV.CPP.6 | SAP(E) | VELPPPVELPPPVELPPP | 98 | 18 | Normal |
| | AAV.CPP.7 | SVM3 | KGTYKKKLMRIPLKGT | 99 | 16 | Low |
| | AAV.CPP.8 | (PPR)3 | PPRPPRPPR | 100 | 9 | Normal |
| | AAV.CPP.9 | (PPR)5 | PPRPPRPPRPPRPPR | 101 | 15 | Low |
| | AAV.CPP.10 | Poly- | RRRRRRRR | 102 | 8 | Low |
| | | arginine | | | | |
| | AAV.CPP.11 | Bipl | VPALR | 1 | 5 | Normal |
| | AAV.CPP.12 | Bip2 | VSALK | 2 | 5 | Normal |
| | AAV.CPP.13 | DPV15 | LRRERQSRLRRERQSR | 103 | 16 | NA |
| | AAV.CPP.14 | HIV-1 Tat | RKKRRQRRR | 104 | 9 | NA |
| Follow-up | AAV.CPP.15 | Bip1.1 | TVPALR (Rat) | 3 | 6 | Normal |
| Screening | AAV.CPP.16 | Bip2.1 | TVSALK (Syn) | 4 | 6 | Normal |
| | AAV.CPP.17 | Bip2.2 | FTVSALK (Syn) | 5 | 7 | Normal |
| | AAV.CPP.18 | Bip2.3 | LTVSALK (Syn) | 6 | 7 | Normal |
| | AAV.CPP.19 | Bip2.4 | KFTVSALK (Syn) | 72 | 8 | Normal |
| | AAV.CPP.20 | Bip2.5 | TFVSALK (Syn) | 7 | 7 | Normal |
| | AAV.CPP.21 | Bip2.6 | TVSALFK (Syn) | 8 | 7 | Normal |
| | AAV.CPP.22 | Bip2.6Rat | TVPALFR (Rat) | 9 | 7 | Normal |

#, SEQ ID NO:
Syn, synthetic

### Example 2. First Round of In Vivo Screening

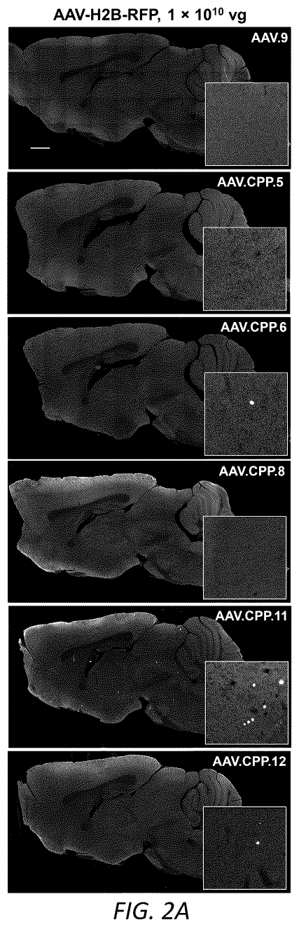

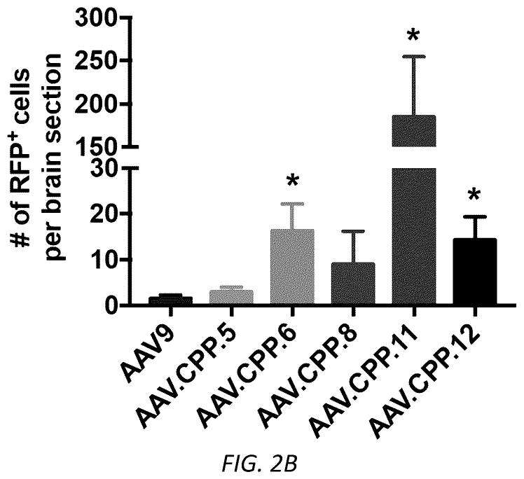

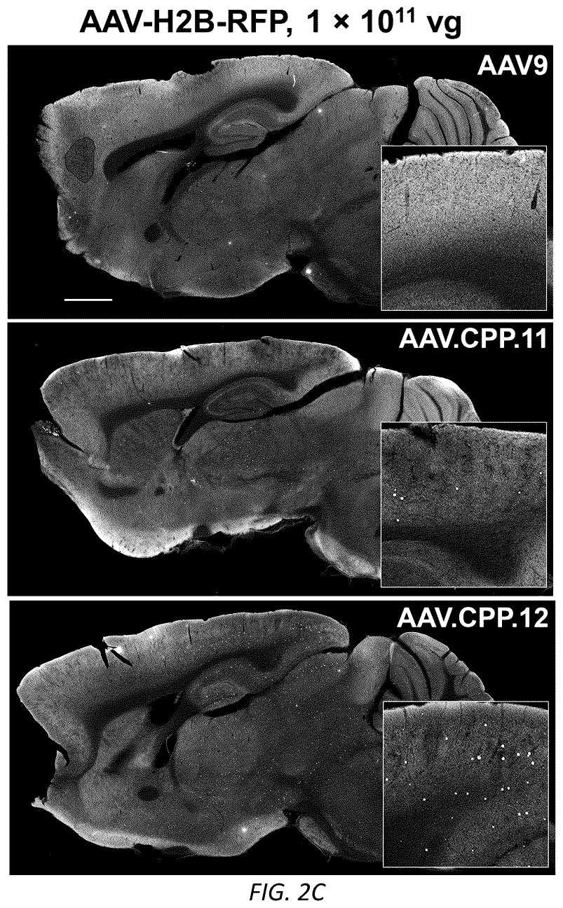

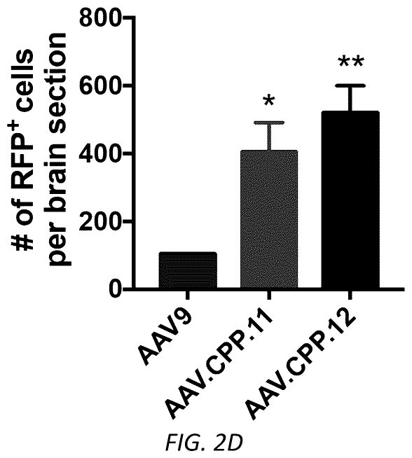

AAVs expressing nuclear RFP (H2B-RFP) were injected intravenously in adult mice with mixed C571BL/6 and BALB/c genetic background. 3 weeks later, brain tissues were harvested and sectioned to reveal RFP-labelled cells (white dots in FIGS. 2A and 2C, quantified in FIGS. 2B and 2D), respectively). CPPs BIP1 and BIP2 were inserted into the capsids of AAV.CPP.11 and AAV.CPP.12, respectively. See Materials and Methods #4-5 for more details.

### Example 3. Optimization of Modified AAV9 Capsids

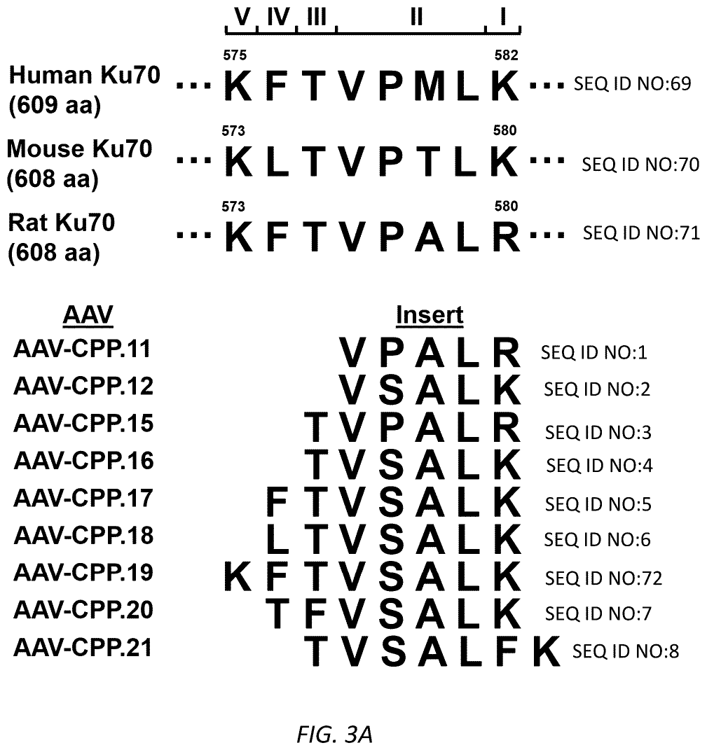

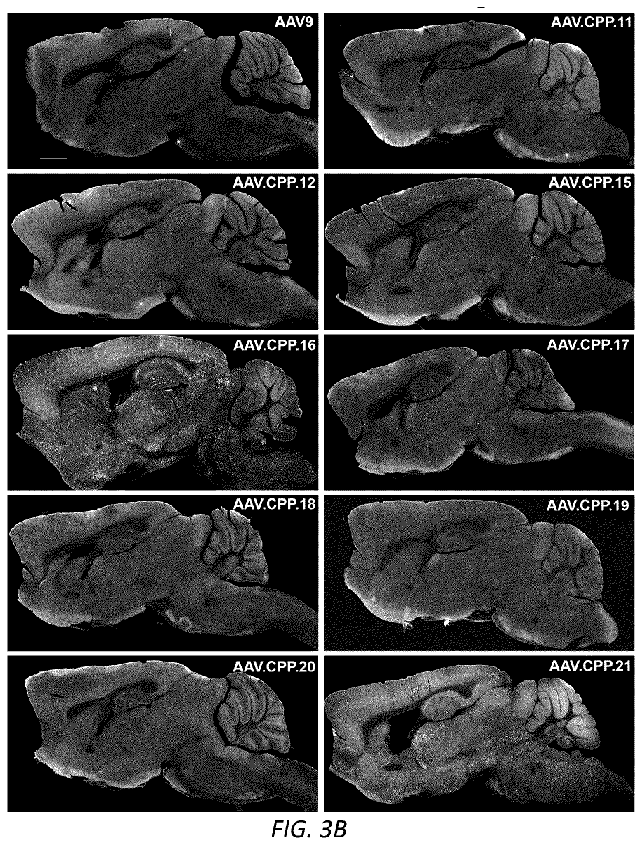

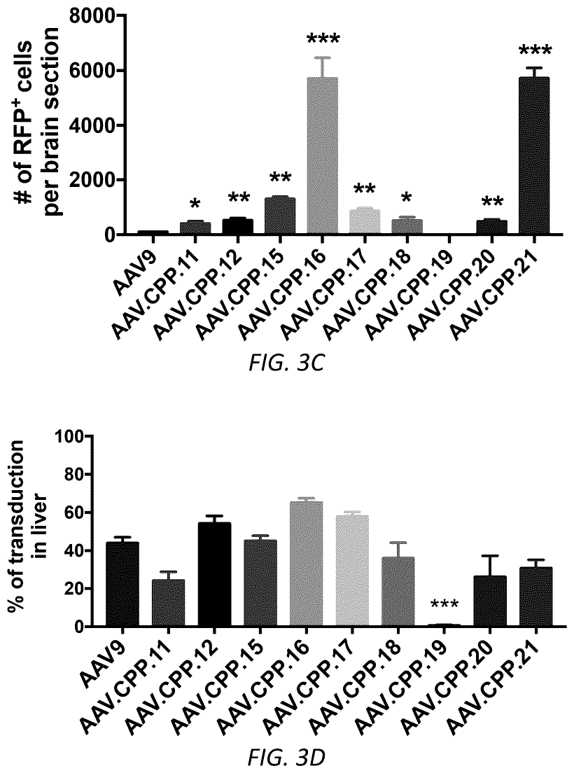

AAV.CPP.11 and AAV.CPP.12 were further engineered by optimizing the BIP targeting sequences. BIP inserts were derived from the protein Ku70 (See FIG. 3A and Material/Methods #1 for full sequence). The BIP sequence VSALK, which is of “synthetic” origin, was chosen as a study focus to minimize potential species specificity of engineered AAV vectors. AAVs were produced and tested separately for brain transduction efficiency as compared with AAV9 (see FIGS. 3B-C). Percentages of cell transduction in the mouse liver 3 weeks after IV injection of some AAV variants delivering the reporter gene RFP are shown in FIG. 3D. See Materials and Methods #1-5 for more details.

### Example 4. In Vitro Model—BBB Permeation Screening

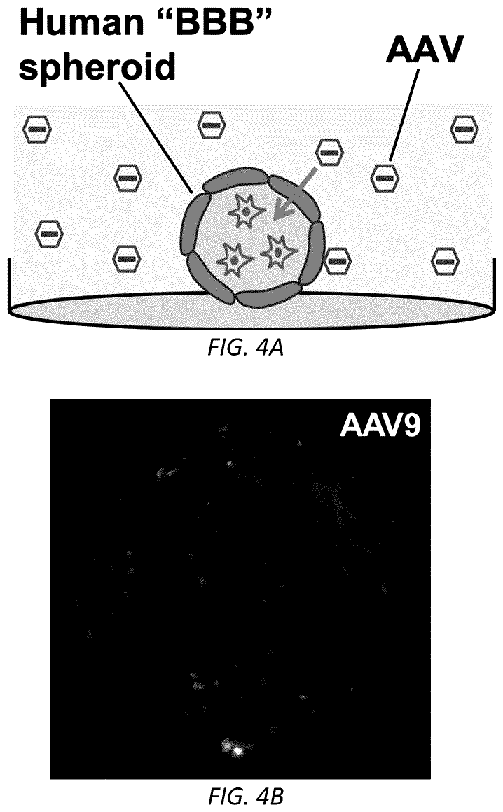

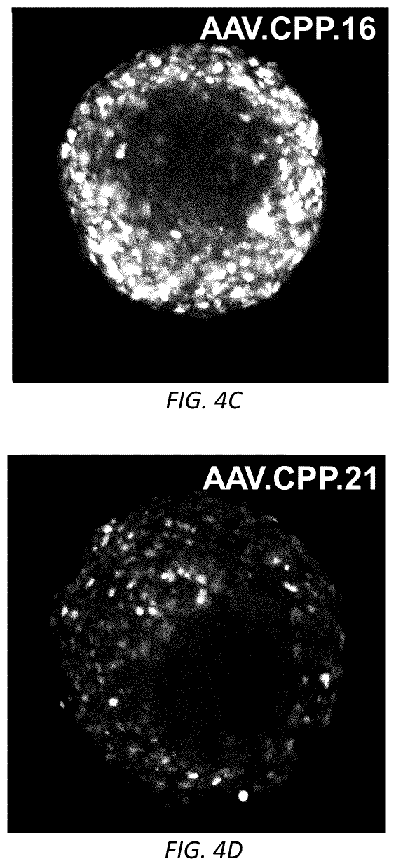

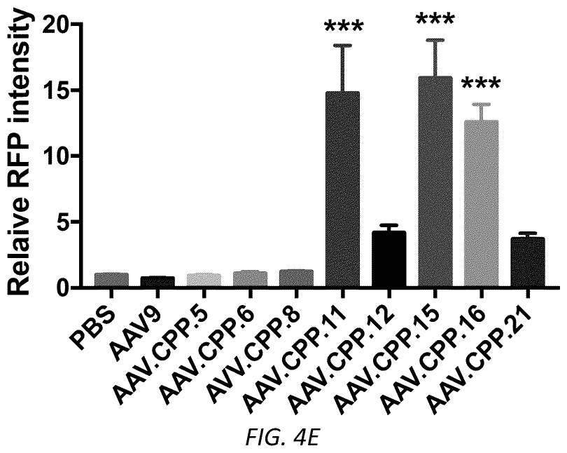

Some of the AAV variants were screened for the ability to cross the human BBB using an in vitro spheroid BBB model. The spheroid contains human microvascular endothelial cells, which form a barrier at the surface, and human pericytes and astrocytes. AAVs carrying nuclear RFP as reporter were assessed for their ability to penetrate from the surrounding medium into the inside of the spheroid and to transduce the cells inside. FIG. 4A shows an experimental schematic. FIGS. 4B-D show results for wt AAV9, AAV.CPP.16, and AAV.CPP.21, respectively, those and other peptides are quantified in FIG. 4E. In this model, peptides 11, 15, 16, and 21 produced the greatest permeation into the spheroids. See Materials and Methods #6 for more details.

### Example 5. In Vivo BBB Permeation Screening

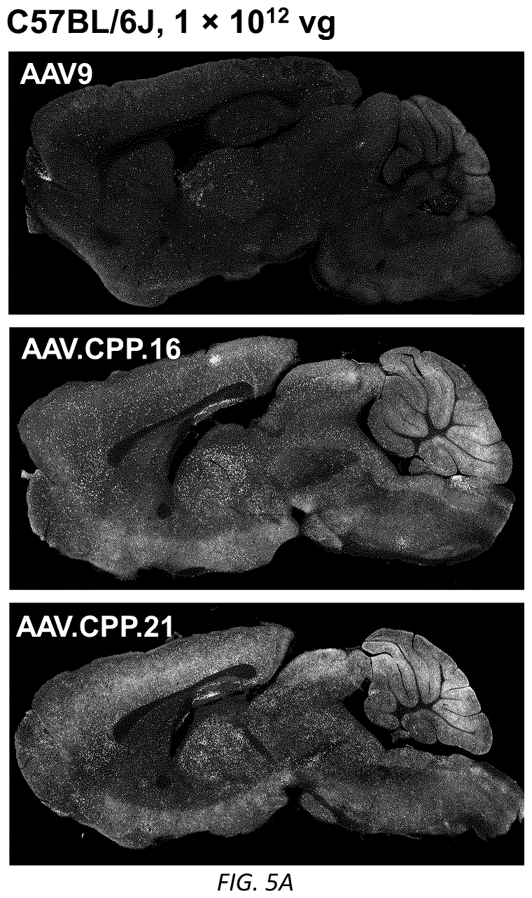

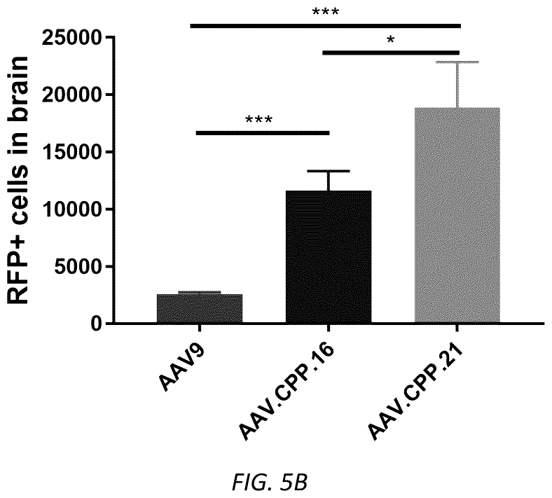

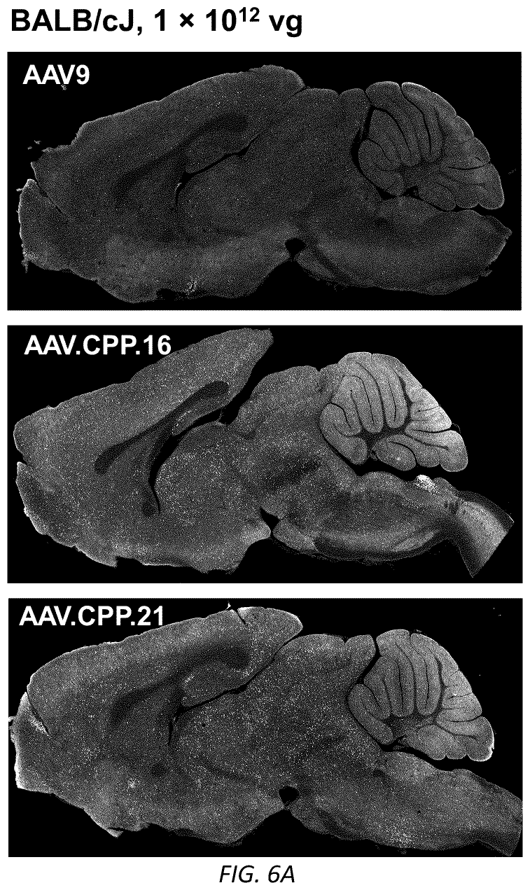

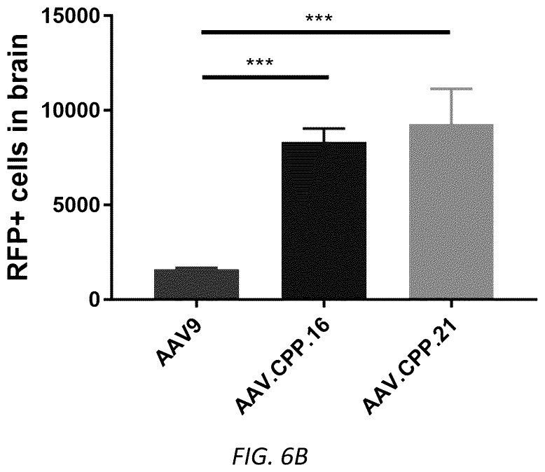

AAV.CPP.16 and AAV.CPP.21 were selected for further evaluation in an in vivo model, in experiments performed as described above for Example 2. All AAVs carried nuclear RFP as reporter. Both showed enhanced ability vs. AAV9 to transduce brain cells after intravenous administration in C57BL/6J adult mice (white dots in brain sections in FIG. 5A, quantified in FIG. 5B) and in BALB/c adult mice (white dots in brain sections in FIG. 6A, quantified in FIG. 6B).

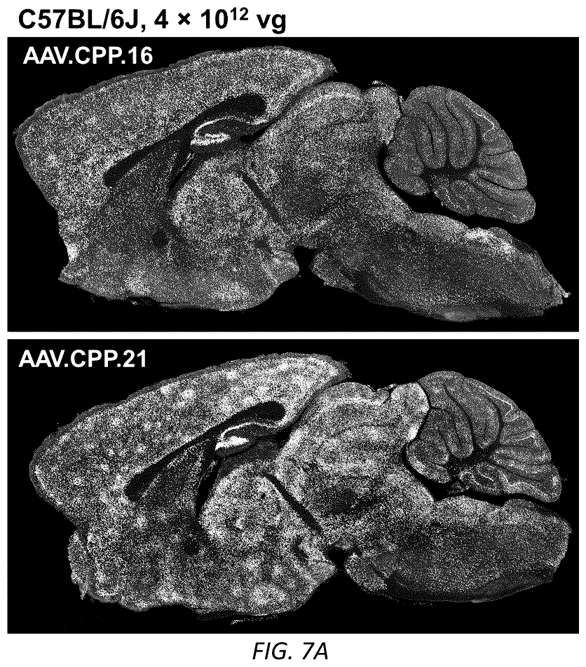

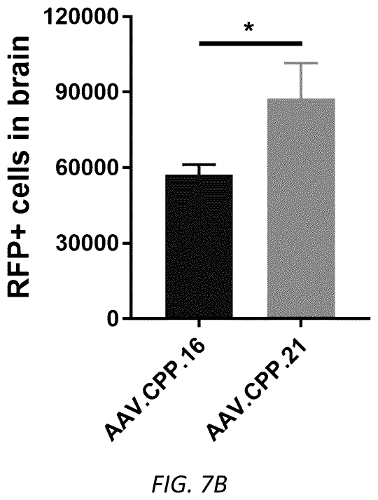

High doses of AAV.CPP.16 and AAV.CPP.21 (4×10¹² vg per mouse, administered IV) resulted in widespread brain transduction in mice. Both AAVs carried nuclear RFP as reporter (white dots in brain sections in FIG. 7A, quantified in FIG. 7B).

### Example 6. In Vivo Distribution of Modified AAVs

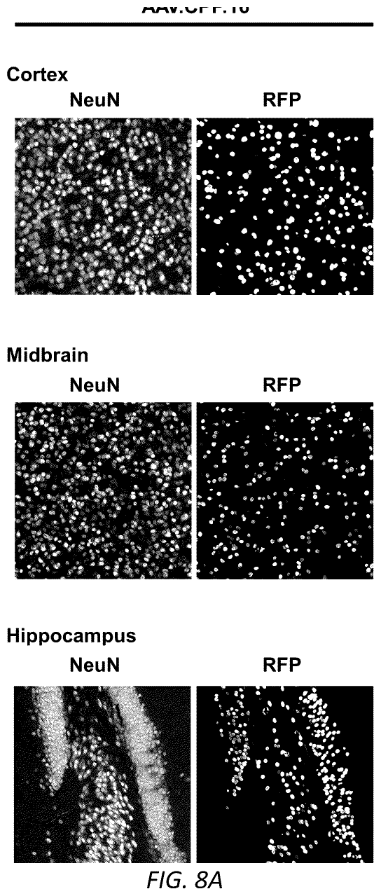

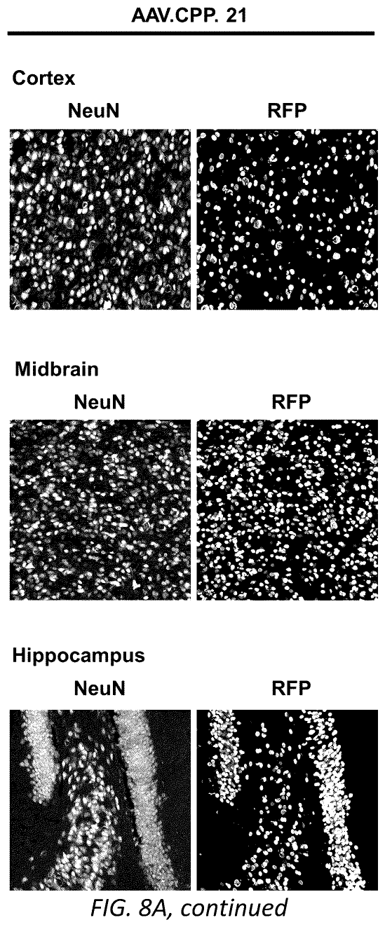

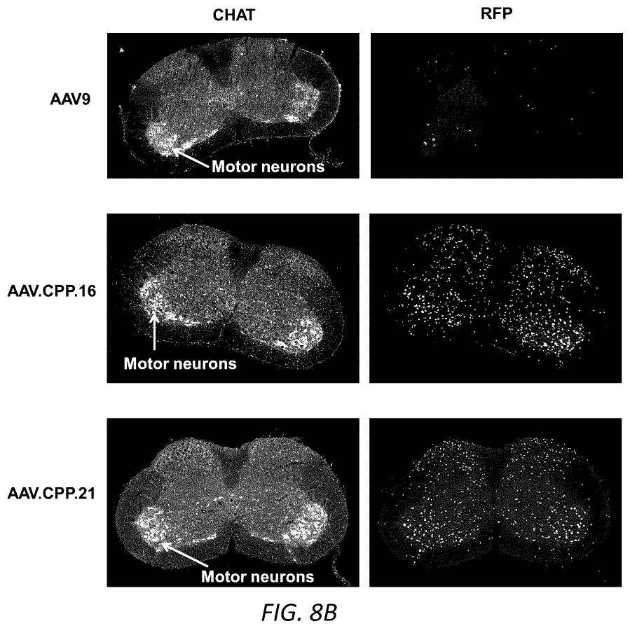

As shown in FIG. 8A, AAV.CPP.16 and AAV.CPP.21 preferentially targeted neurons (labeled by a NeuN antibody) across multiple brain regions in mice including the cortex, midbrain and hippocampus. Both AAVs carried nuclear RFP as a reporter.

AAV.CPP.16 and AAV.CPP.21 also showed enhanced ability vs. AAV9 in targeting the spinal cord and motor neurons in mice. All AAVs carry nuclear RFP as reporter and were administered intravenously into neonate mice (4×10¹⁰ vg). Motor neurons were visualized using CHAT antibody staining. Co-localization of RFP and CHAT signals in FIG. 8B suggested specific transduction of the motor neurons.

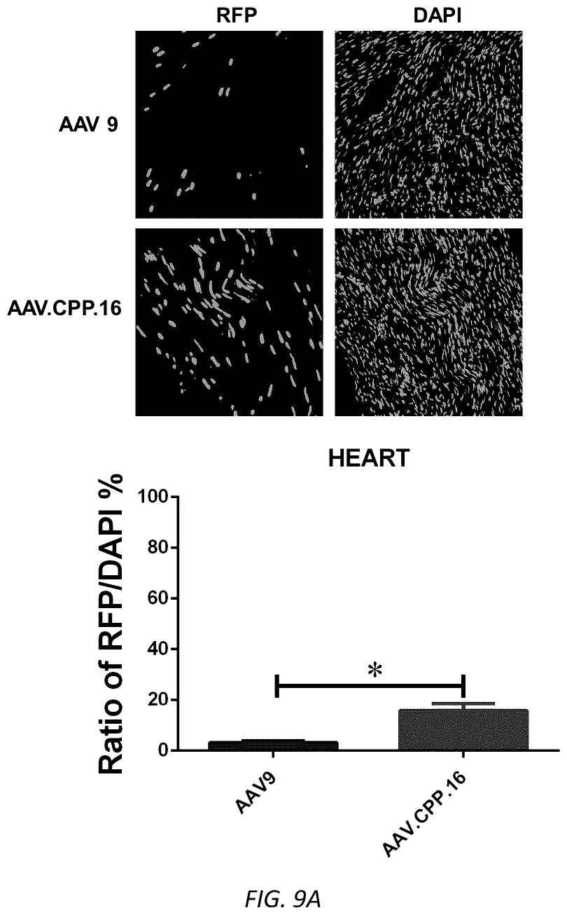

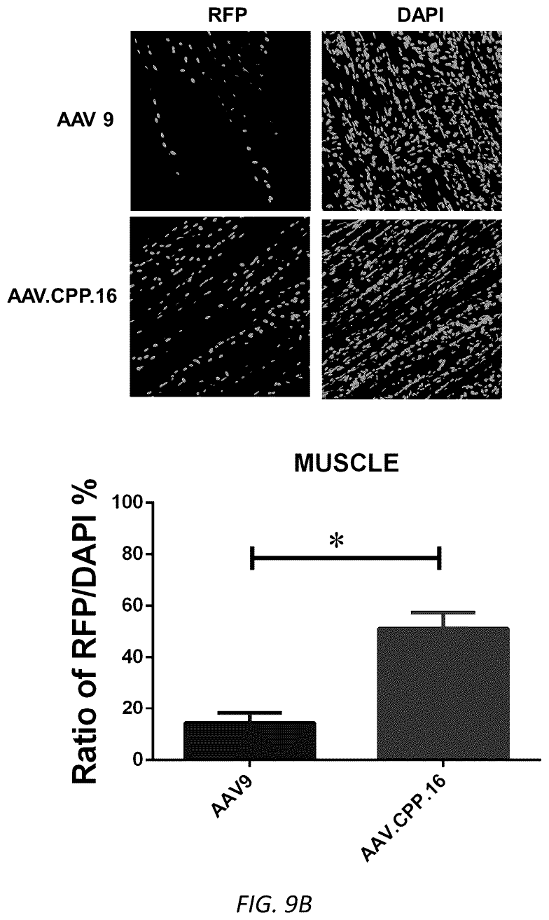

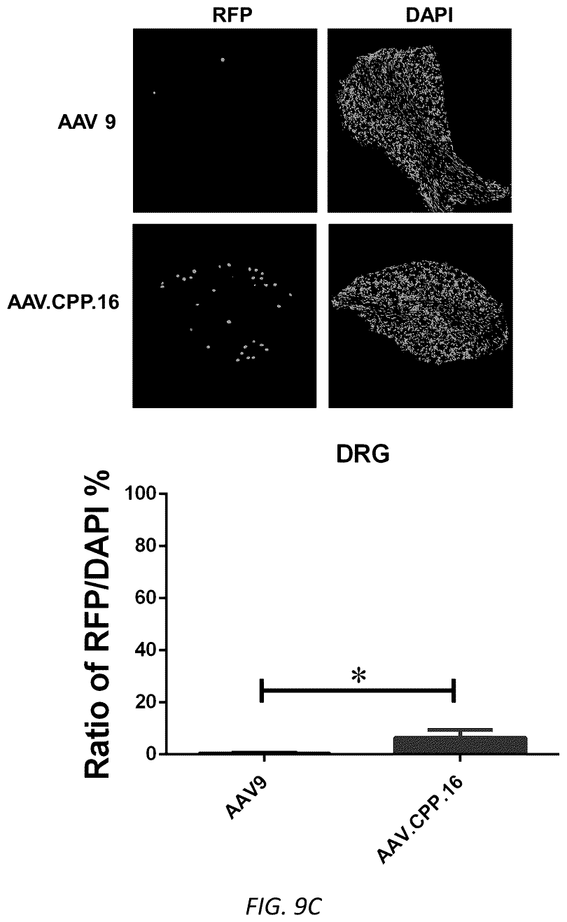

The relative abilities of AAV-CAG-H2B-RFP and AAV.CPP.16-CAG-H2B-RFP to transduce various tissues in mice was also evaluated. 1×10¹¹ vg was injected intravenously. The number of cells transduced was normalized to the number of total cells labeled by DAPI nuclear staining. The results showed that AAV.CPP.16 was more efficient than AAV9 in targeting heart (FIG. 9A); skeletal muscle (FIG. 9B), and dorsal root ganglion (FIG. 9C) tissue in mice.

### Example 7. BBB Permeation in a Non-Human Primate Model

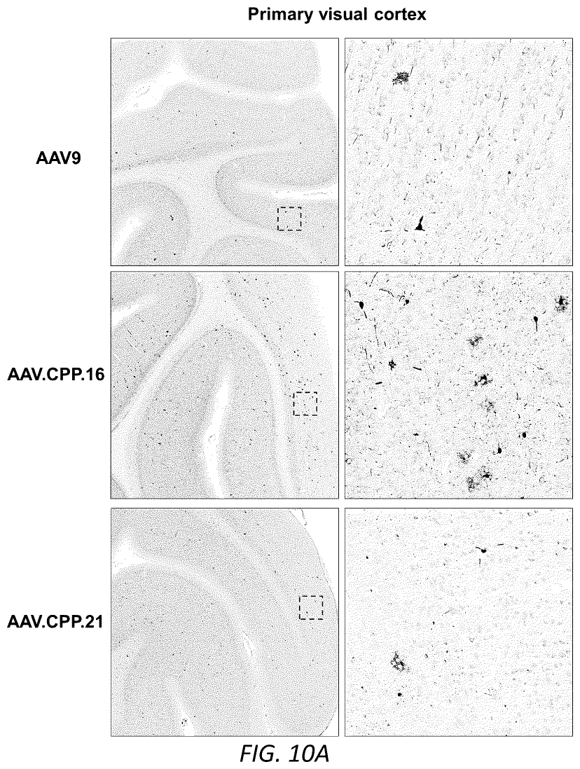

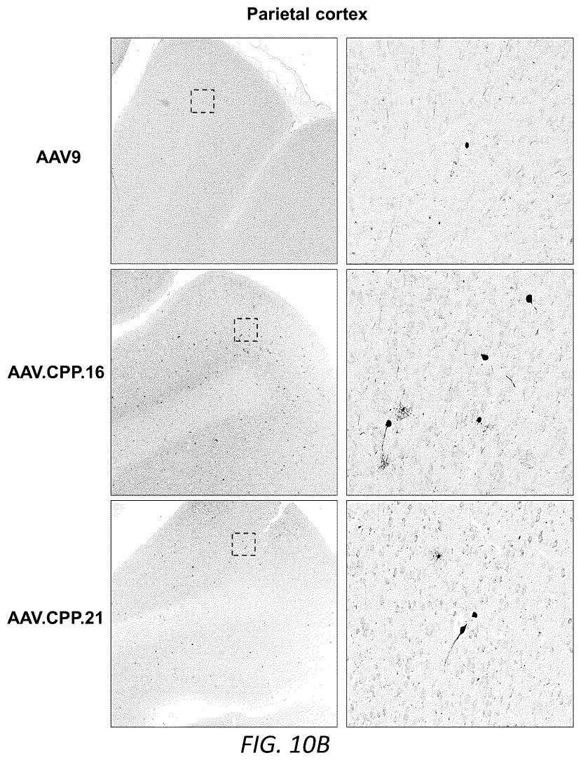

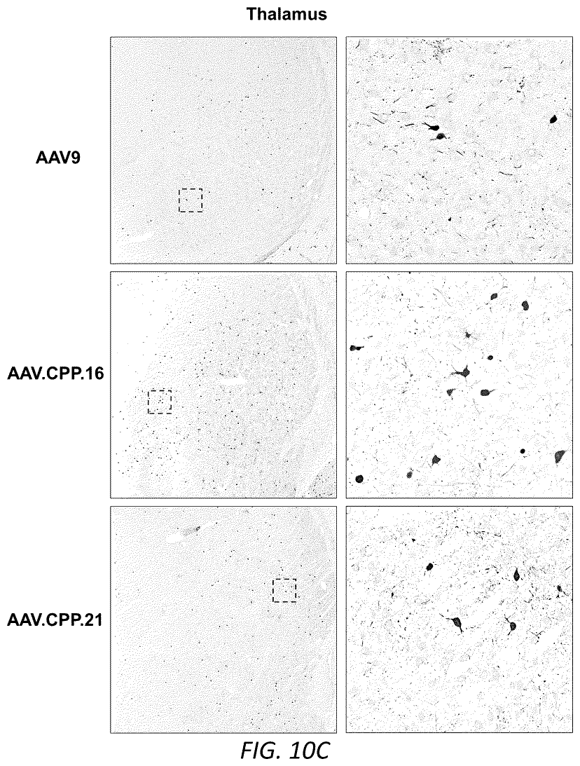

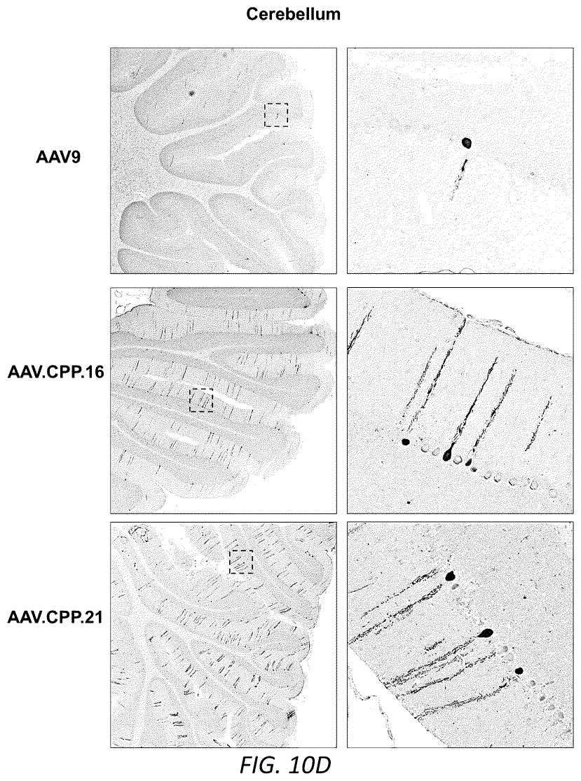

2×10¹³ vg/kg AAVs-CAG-AADC (as reporter gene) were injected intravenously into 3-month-old cynomolgus monkeys. AAV-transduced cells (shown in black) were visualized using antibody staining against AADC. As shown in FIGS. 10A-D, AAV.CPP.16 and AAV.CPP.21 showed enhanced ability vs. AAV9 to transduce brain cells after intravenous administration in non-human primates. AAV.CPP.16 transduced significantly more cells then wt AAV9 in the primary visual cortex (FIG. 10A), parietal cortex (FIG. 10B), thalamus (FIG. 10C), and cerebellum (FIG. 10D). See Materials and Methods #7-8 for more details.

### Example 8. AAV.CPP.16 and AAV.CPP.21 do not Bind to LY6A

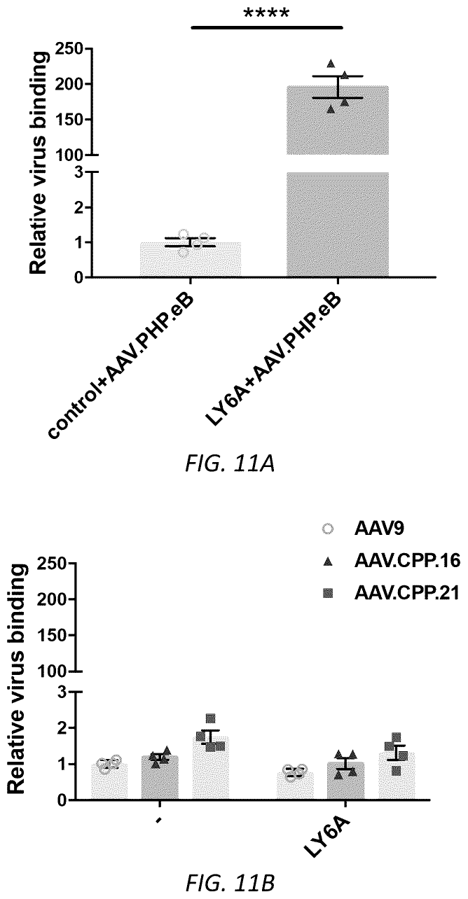

LY6A serves as a receptor for AAV.PHP.eB and mediates AAV.PHP.eB's robust effect in crossing the BBB in certain mouse strains. Over-expressing mouse LY6A in cultured 293 cells significantly increased binding of AAV.PHP.eB to the cell surface (see FIG. 11A). On the contrary, over-expressing LY6A does not increase viral binding for AAV9, AAV.CPP.16 or AAV.CPP.21 (see FIG. 11B). This suggests AAV.CPP.16 or AAV.CPP.21 does not share LY6A with AAV.PHP.eB as a receptor. See Materials and Methods #9 for more details.

### Example 9. Delivering Therapeutic Proteins to the Brain Using AAV.CPP.21

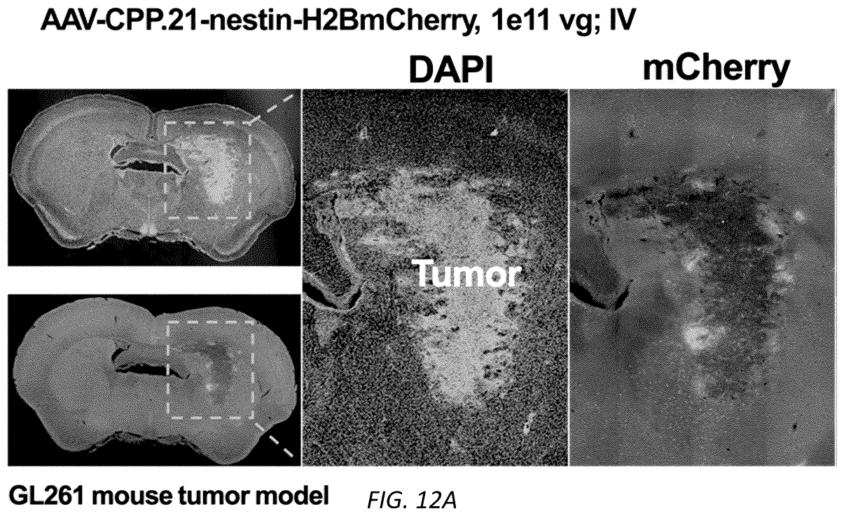

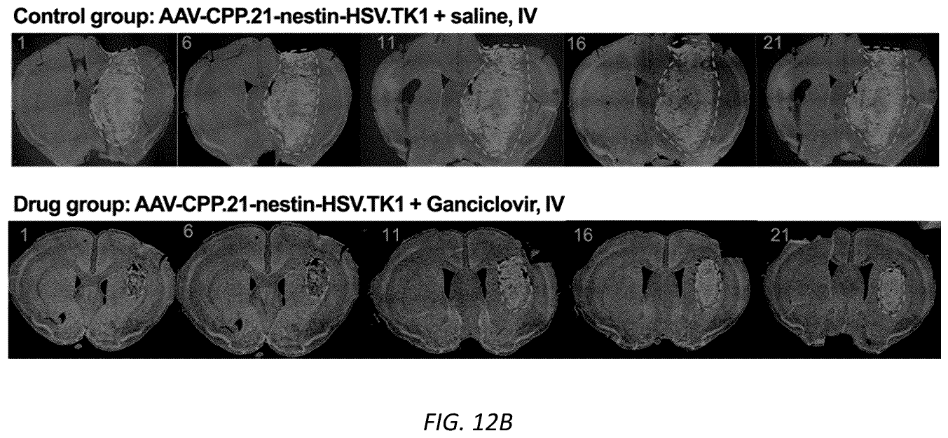

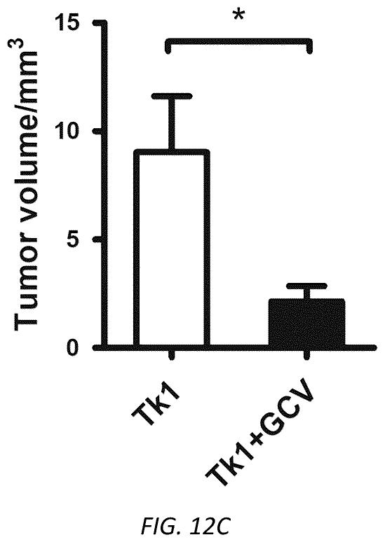

AAV.CPP.21 was used to systemically deliver the “suicide gene” HSV.TK1 in a mouse model of brain tumor (Materials and Methods #10). HSV.TK1 turns the otherwise “dormant” ganciclovir into a tumor-killing drug. Intravenously administered AAV.CPP.21-H2BmCherry (FIG. 12A, bottom left and middle right panel) was shown to target tumor mass, especially the tumor expanding frontier. As shown in FIGS. 12B-C, using AAV.CPP.21 to systemically deliver the “suicide gene” HSV.TK1 resulted in shrinkage of brain tumor mass, when combined with the pro-drug ganciclovir. These results show that AAV.CPP.21 can be used to systemically deliver a therapeutic gene into brain tumor. See Materials and Methods #10 for more details.

### Example 10. Intracerebral Administration of AAV.CPP.21

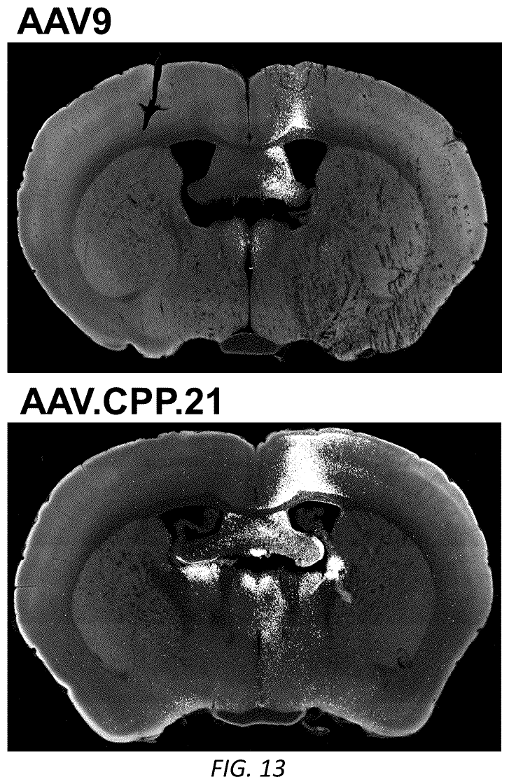

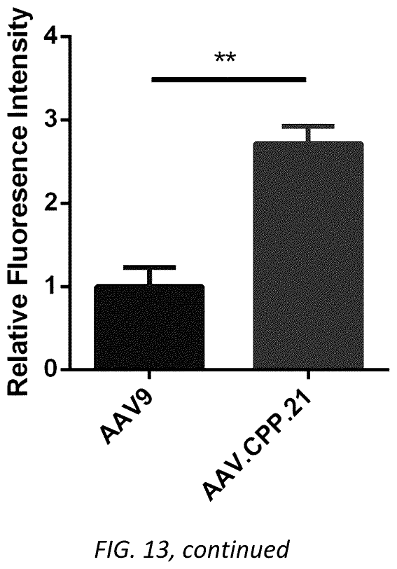

In addition to systemic administration (such as in Example 2), an AAV as described herein was administered locally into the mouse brain. Intracerebral injection of AAV9-H2B-RFP and AAV.CPP.21-H2B-RFP (FIG. 13) resulted in more widespread and higher-intensity RFP signal in AAV.CPP.21-treated brain sections vs. AAV9-treated ones. See Materials and Methods #4 for more details.

### Example 11. Systemic Delivery of AAV.CPP.16 to the Glioblastoma Tumor Microenvironment

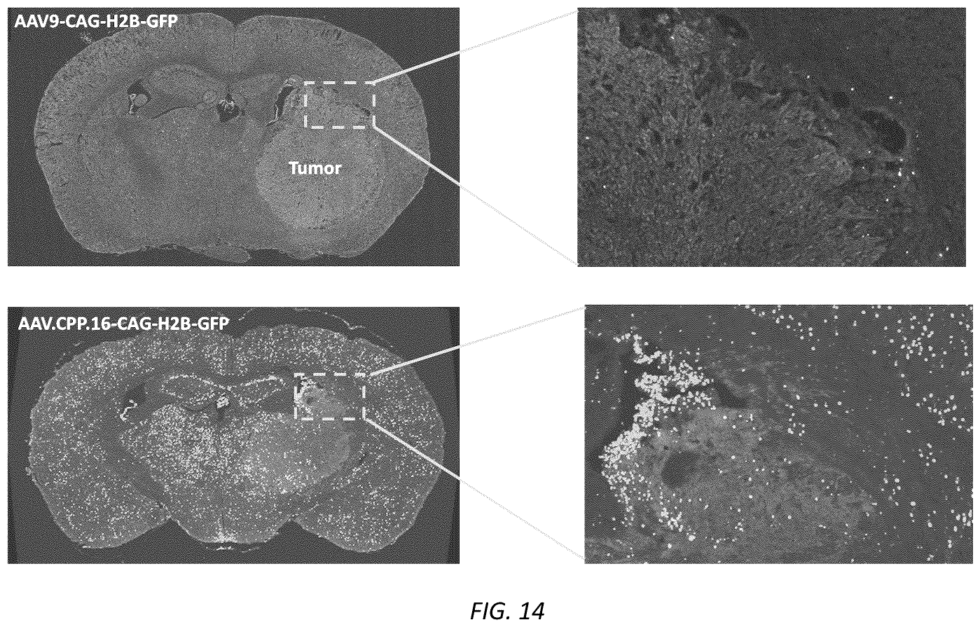

Using systemic administration (such as in Example 2), delivery of an AAV as described herein into the brains of an orthotopic, immunocompetent mouse glioblastoma model (GL261 model). (as described in Materials and Methods #10). As shown in FIG. 14, AAV.CPP16 far outperformed AAV9, with significant delivery both to tumors and to the surrounding microenvironment.

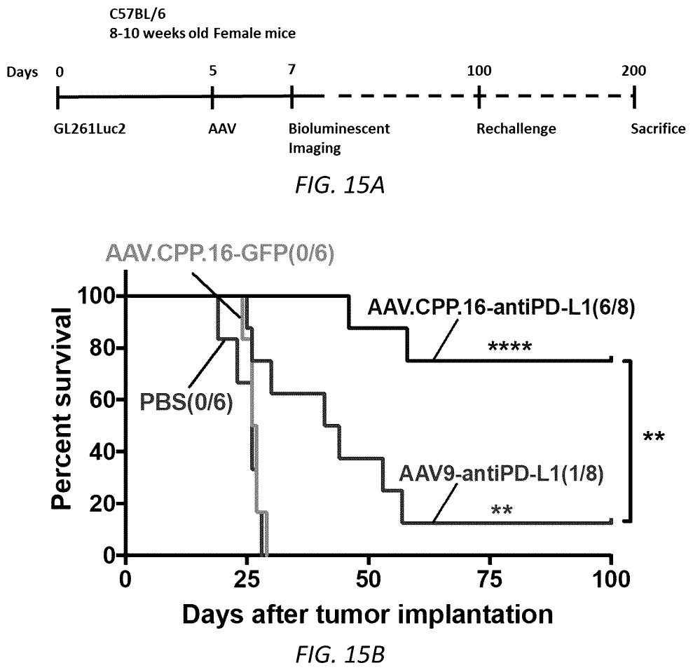

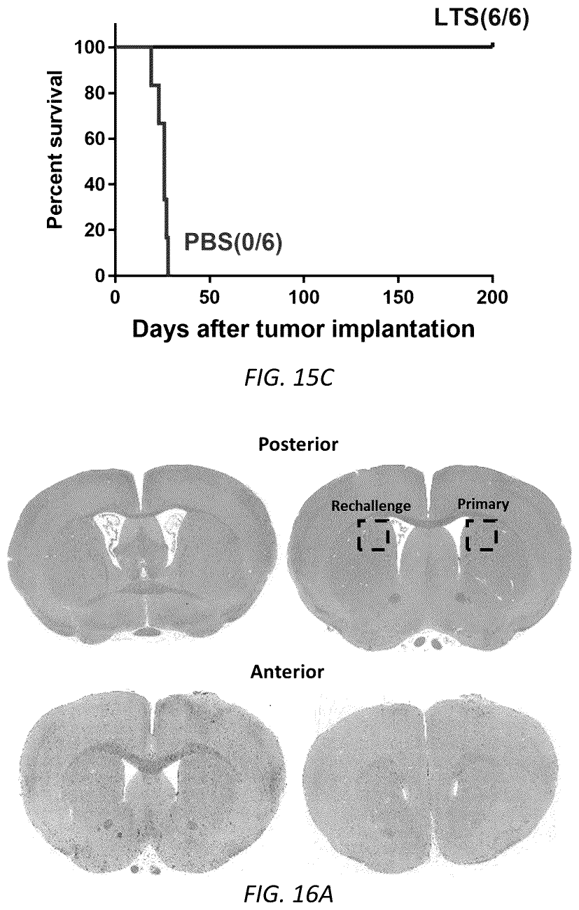

To determine whether this increased efficiency of delivery would translate to improved therapeutic efficacy, various treatments were administered to the mouse GBM model; FIG. 15A provides a schematic of the experimental protocol. The results, shown in FIGS. 15B-C, demonstrated that AAV.CPP.16-antiPD-L1 mediated immunotherapy significantly prolonged survival in the murine GBM model. As shown in FIG. 15B, 1 of 8 mice treated with AAV9-antiPD-L1 survived long term, while 6/8 mice treated with the AAV.CPP.16-antiPD-L1 survived long term (longer than 100 days). FIG. 15C shows that all 6 of the long-term survivors (five treated with AAV.CPP.16-antiPD-L1 plus one treated with AAV9-antiPD-L1; one of the long-term survivors treated with AAV.CPP.16-antiPD-L1 died during re-challenging surgery due to technical reasons) were still alive 200 days after tumor implantation. Thus, intravenous injection of AAV.CPP.16 expressing an antibody targeting the mouse PD-L1 eradicated GBM tumors in 75% of the mice, whereas untreated mice died within a month of tumor implantation.

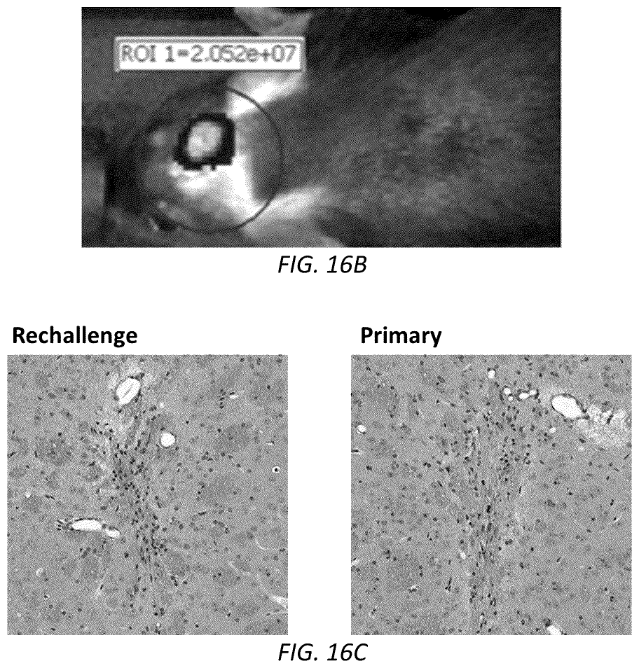

The long-term surviving mice were sacrificed at 200 days, and their brains examined. As shown in FIG. 16A, no evidence of tumors remained. FIG. 16B shows a bioluminescent image taken of one of the mice that had extended survival, showing that at 7 days post implant the tumor cells were present. FIG. 16C shows that the initial tumor implantation is devoid of residual tumor and only gliotic scar tissue remains, indicating complete tumor eradication.

Furthermore, immunohistochemistry showed that CB8+ cytotoxic T cells were also present in the GBM tumor site, further evidence for an immune reaction.

### Example 12. Expression of HA-Tagged AntiPD-L1 Antibody in GBM Tumor

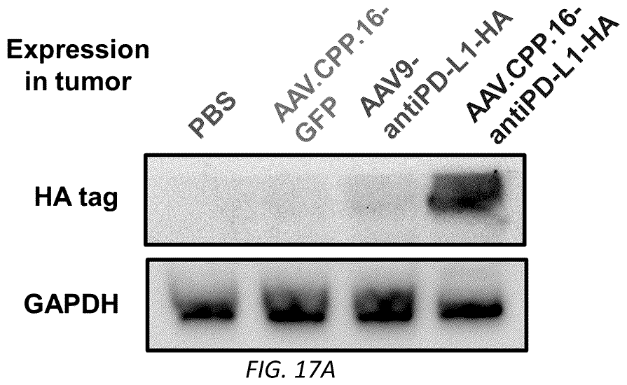

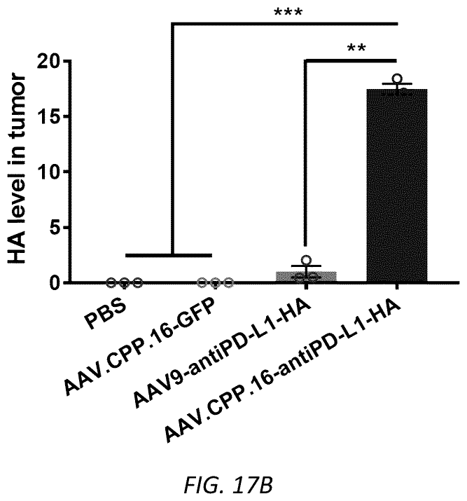

Expression of HA-tagged antiPD-L1 antibody in GBM tumor as measured by Western blotting is shown in FIGS. 17A-17B. AAVs of 1e12 vg or PBS were injected intravenously 5 days after tumor implantation in mice. Tumor tissues were harvested 14 days after IV injection. The intensities of HA tag staining (FIG. 17A) were quantified as measurement of antiPD-L1 antibody expression (FIG. 17B).

### REFERENCES

1. Stupp, R. et al. Radiotherapy plus concomitant and adjuvant temozolomide for glioblastoma. The New England journal of medicine 352, 987-996 (2005).
2 Ostrom, Q. T. et al. CBTRUS Statistical Report: Primary brain and other central nervous system tumors diagnosed in the United States in 2010-2014.

Neuro-oncology 19, v1-v88 (2017).
3 Brennan, C. W. et al. The somatic genomic landscape of glioblastoma. Cell 155, 462-477 (2013).
4 Lim, M., Xia, Y., Bettegowda, C. & Weller, M. Current state of immunotherapy for glioblastoma. Nature reviews. Clinical oncology 15, 422-442 (2018).
5 Perry, A. & Wesseling, P. Histologic classification of gliomas. Handbook of clinical neurology 134, 71-95 (2016).
6 Wen, P. Y. & Kesari, S. Malignant gliomas in adults. The New England journal of medicine 359, 492-507 (2008).
7 Jain, R. K. et al. Angiogenesis in brain tumours. Nature reviews. Neuroscience 8, 610-622 (2007).
8 Deverman, B. E., Ravina, B. M., Bankiewicz, K. S., Paul, S. M. & Sah, D. W. Y. Gene therapy for neurological disorders: progress and prospects. Nature reviews. Drug discovery (2018).
9 Hudry, E. & Vandenberghe, L. H. Therapeutic AAV Gene Transfer to the Nervous System: A Clinical Reality. Neuron 101, 839-862 (2019).
10 Batista, A. R. et al. Ly6a differential expression in BBB is responsible for strain specific CNS transduction profile of AAV-PHP.B. Human gene therapy (2019).
11 Hordeaux, J. et al. The GPI-Linked Protein LY6A Drives AAV-PHP.B Transport across the Blood-Brain Barrier. Molecular therapy: the journal of the American Society of Gene Therapy 27, 912-921 (2019).
12 Huang, Q. et al. Delivering genes across the blood-brain barrier: LY6A, a novel cellular receptor for AAV-PHP.B capsids. PloS one 14, e0225206 (2019).
13 Hordeaux, J. et al. The Neurotropic Properties of AAV-PHP.B Are Limited to C57BL/6J Mice. Molecular therapy: the journal of the American Society of Gene Therapy 26, 664-668 (2018).
14 Matsuzaki, Y. et al. Intravenous administration of the adeno-associated virus-PHP.B capsid fails to upregulate transduction efficiency in the marmoset brain. Neuroscience letters 665, 182-188 (2018).
15 Nakashima, H. et al. Modeling tumor immunity of mouse glioblastoma by exhausted CD8(+) T cells. Scientific reports 8, 208 (2018).
16 Reul et al., Tumor-Specific Delivery of Immune Checkpoint Inhibitors by Engineered AAV Vectors. Front Oncol. 2019 Feb. 14; 9:52.
17 Engeland et al., CTLA-4 and PD-L1 Checkpoint Blockade Enhances Oncolytic Measles Virus Therapy. Molecular Therapy 22(11):1949-1959 November 2014.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1.-3. (canceled)

4. A method of delivering an immunotherapy agent to treat a cancer in a subject, the method comprising administering to the subject an adeno-associated virus (AAV) vector comprising (i) a capsid protein comprising a targeting sequence that comprises at TV[S/p][A/m/t/]L (SEQ ID NO:80), and (ii) a transgene encoding the immunotherapy agent.

5.-6. (canceled)

7. The method of claim 4, wherein the subject is a human subject.

8. The method of claim 4, wherein the AAV is AAV9.

9. The method of claim 8, wherein the AAV9 comprises AAV9 VP1.

10. The method of claim 9, wherein the targeting sequence is inserted in a position corresponding to amino acids 588 and 589 of AAV9 VP1 comprising SEQ ID NO:85.

11. The method of claim 4, wherein the AAV is administered by parenteral delivery intracerebral delivery, or intrathecal delivery.

12. The method of claim 11, wherein the parenteral delivery is via intravenous, intraarterial, subcutaneous, intraperitoneal, or intramuscular delivery.

13. The method of claim 12, wherein the intrathecal delivery is via lumbar injection, cisternal magna injection, or intraparenchymal injection.

14. The method of claim 4, further comprising administering chemotherapy, radiation, and/or surgical resection to the subject.

15. The method of claim 14, wherein the chemotherapy comprises temozolamide, lomustine, or a combination thereof.

16. The method of claim 4, wherein the immunotherapy agent is an immune checkpoint inhibitor.

17. The method of claim 16, wherein the immune checkpoint inhibitor is an antibody or antigen binding fragment which binds PD-1, PD-L1, CLTA-4, or CD137.

18. The method of claim 17, wherein the antibody or antigen binding fragment binds PD-1.

19. The method of claim 4, wherein the cancer is in the brain of the subject.

20. The method of claim 19, wherein the cancer is a glioma, a metastases from lung, breast, melanoma or colon cancer, a meningioma, a pituitary adenoma, or an acoustic neuroma.

21. The method of claim 20, wherein the glioma comprises glioblastoma multiforme (GBM).

22. The method of claim 4, wherein the AAV vector comprises a promoter sequence, an enhancer sequence, a 5′ untranslated region (UTR), a 3′ UTR, a polyadenylation site, an insulator sequence, or a combination thereof.

23. The method of claim 22, wherein the promoter sequence is a pan-cell type promoter selected from the group consisting of a cytomegalovirus (CMV) promoter, a beta glucuronidase (GUSB) promoter, an ubiquitin C (UBC) promoter, and a rous sarcoma virus (RSV) promoter.

24. The method of claim 22, wherein the promoter is selected from the group consisting of a neuronal nuclei (NeuN) promoter, a glial fibrillary acidic protein (GFAP) promoter, a MeCP2 promoter, an adenomatous polyposis coli (APC) promoter, an ionized calcium binding adapter molecule 1 (Iba-1) promoter, a synapsin I (SYN) promoter, a calcium/calmodulin-dependent protein kinase II promoter, a tubulin alpha I promoter, a neuron-specific enolase promoter, and a platelet-derived growth factor beta chain promoter.
